(19) United States
(12) Patent Application Publication
(10) Pub. No.: US 2025/0152643 A1
(43) Pub. Date: May 15, 2025

(54) COMBINATIONS OF CHECKPOINT INHIBITORS AND ONCOLYTIC VIRUS FOR TREATING CANCER

(71) Applicants: Regeneron Pharmaceuticals, Inc., Tarrytown, NY (US); Vyriad, Inc., Rochester, MN (US)

(72) Inventors: Giuseppe Gullo, White Lake, NY (US); Stephen J. Russell, Rochester, MN (US)

(21) Appl. No.: 18/839,107

(22) PCT Filed: Feb. 16, 2023
(86) PCT No.: PCT/US23/62710

§ 371 Date: Aug. 16, 2024

Related U.S. Application Data
(60) Provisional application No. 63/365,288, filed on May 25, 2022.
(60) Provisional application No. 63/268,139, filed on Feb. 17, 2022.

(51) Int. Cl. A61K 35/768; A61K 45/06; C07K 14/565; C07K 16/28

(52) U.S. Cl. CPC A61K 35/768; A61K 45/06; C07K 14/565; C07K 16/2818; C07K 2317/565; C12N 2760/20222; C12N 2760/20233

(57) ABSTRACT

The present disclosure relates to methods for inhibiting the growth of a tumor in a patient in need thereof. The methods comprise administering to the patient an oncolytic virus in combination with a therapeutically effective amount of a PD-1 inhibitor (e.g., cemiplimab or a bioequivalent thereof) and optionally, a therapeutically effective amount of a CTLA-4 inhibitor (e.g., ipilimumab or a bioequivalent thereof). In some embodiments, the administration of the combination results in enhanced tumor inhibition as compared to administration of either antibody or the virus alone. In some embodiments, the oncolytic virus is a recombinant vesicular stomatitis virus (VSV), such as VV1.

### FIELD

The present disclosure relates generally to combination therapies for cancer treatment with oncolytic viruses and checkpoint inhibitors such as programmed death 1 (PD-1) pathway inhibitors and/or cytotoxic T-lymphocyte antigen-4 (CTLA-4) inhibitors.

### BACKGROUND

VV1 (also known as VSV-IFN3-NIS) is derived from vesicular stomatitis virus (VSV), a bullet-shaped, negative-sense ribonucleic acid (RNA) virus with low human seroprevalence; it is engineered to replicate selectively in and kill human cancer cells. The virus encodes human interferon β (IFNβ) to boost anti-tumoral immune responses and increase tumor specificity, plus the thyroidal sodium iodide symporter (NIS) to allow imaging of virus spread. VSV-IFNβ-NIS is synergistic with different anti-PD-(L)1 antibodies in several tumor models.

VV1 is a live RNA virus engineered to express both the human IFNβ gene and the NIS gene. The virus was constructed by inserting the gene for IFNβ downstream of the M gene and the NIS gene (cDNA) downstream of the gene for the G protein into a full-length infectious molecular clone of an Indiana strain VSV [Naik 2012]. This virus is not a vaccine. VSV-IFNβ-NIS propagates on BHK cells with similar kinetics to the parental strain of virus and can be grown to high titers. It propagates selectively in human cancer cells since many of them cannot mount an effective antiviral response mediated via the IFNβ pathway. However, IFNβ production from infected cells will serve to protect noncancer cells from the effects of the virus. As a result, the virus is directly cytopathic to tumor cells leading to rapid lysis with amplification of the virus [Barber 2004; Lichty 2004)]. Preclinical studies demonstrate that IV treatment with oncolytic VSV selectively targets tumor cells, inducing an acute inflammatory response and IT immune infiltration [Obuchi 2003; Willmon 2009; Saloura 2010; Naik 2012; Kurisetty 2014; Patel 2015].

VSV-IFNβ-NIS-infected tumor cells also express NIS, a membrane ion channel that actively transports iodide into cells. Radioiodine uptake by cells expressing NIS provides the basis for in vivo imaging with 99mTc pertechnetate or radioiodine I-123 that can reveal the time dependent profile of VSV-IFNβ-NIS gene expression and the location of VSV-IFNβ-NIS infected cells during virus spread and elimination [Naik 2012]. F18-tetrafluoroborate (F18-TFB) PET imaging technology can also be used to track the virus' replication and spread [Miller 2016]. More recently, the safety and feasibility of using F18-TFB PET imaging was demonstrated in healthy human subjects to locate tissues that physiologically express NIS [Jiang 2017].

Programmed death 1 (PD-1) plays an important role in autoimmunity, tumor immunity and infectious immunity, and is thus an ideal target for immunotherapy. Blocking PD-1 with antagonists, including monoclonal antibodies, has been studied in treatments of cancer and chronic viral infections. Blockade of PD-1 is also an effective and well tolerated approach to stimulating the immune response, and has achieved therapeutic advantage against various human cancers, including melanoma, renal cell cancer, and non-small cell lung cancer (NSCLC). (Sheridan 2012, Nat. Biotechnol., 30:729-730; Postow et al., 2015, J Clin Oncol, 33:1974-1982; Chen et al., 2013, Nat. Rev. Immunol., 13:227-242; Riley, 2009, Immunol. Rev., 229:114-125; Dong et al., 1999, Nature Med., 5(12):1365-1369; Zou, 2008, Nat. Rev. Immunol., 8:467-77; Ribas 2012, NEJM 366:2517-2519; Watanabe et al., 2012, Clin. Dev. Immunol. Vol. 2012, Article ID: 269756; Wang et al., 2013, J. Viral Hep., 20:27-39; Flies et al., 2011, Yale J. Biol. Med., 84:409-421; Pardoll, 2012, Nature, 12:252-264; Freeman, 2008, PNAS, 105:10275-10276; Francisco et al., 2010, Immunol. Rev., 236:219-242). Monoclonal antibodies to PD-1 are known in the art and have been described, for example, in U.S. Pat. Nos. 9,987,500, 8,008,449, 8,168,757, US 20110008369, US 20130017199, US 20130022595, WO 2006121168, WO 20091154335, WO 2012145493, WO 2013014668, WO 2009101611, EP 2262837, and EP 2504028. Cemiplimab (also known as REGN2810; LIBTAYO®), for example, is a high-affinity, fully human, hinge-stabilized IgG4P antibody directed to the PD-1 receptor that potently blocks the interaction of PD-1 with its ligands, PD-L1 and PD-L2.

Blockade of cytotoxic T-lymphocyte associated antigen-4 (CTLA-4) has been shown to increase the number of activated T cells, allowing expansion of tumor specific cytotoxic T cells. CTLA-4 is a type I transmembrane T cell inhibitory checkpoint receptor expressed on conventional and regulatory T cells. CTLA-4 negatively regulates T cell activation by outcompeting the stimulatory receptor CD28 from binding to its shared natural ligands, B7-1 (CD80) and B7-2 (CD86). Ipilimumab is a recombinant humanized monoclonal antibody that binds to CTLA-4.

Lung cancer is the leading cause of cancer deaths worldwide [Bray 2018]. In the US, it is estimated that approximately 228,000 patients will be diagnosed with lung cancer and 80 to 85% of these cases will be of NSCLC histology in 2019 [ACS 2019d]. The etiologic association of NSCLC with exposure to tobacco carcinogens is of particular relevance for cancer immunotherapy, as current approaches targeting immune checkpoints exhibit increased responses in tumors with a high number of somatic mutations, as is the case in smoking-induced NSCLC. Conversely, immune checkpoint blockade has been significantly less successful in never-smokers, including a majority of anaplastic lymphoma kinase (ALK)-rearranged or epidermal growth factor receptor (EGFR)-mutated NSCLC [Garassino 2017]. Most patients with NSCLC present at an advanced stage of the disease and are symptomatic at the time of diagnosis, with a related poor prognosis and no curative options. In stage IV, systemic palliative treatment is recommended, including cytotoxic chemotherapy, immune checkpoint inhibitors (CPIs), and a series of targeted agents in selected molecularly defined subsets of NSCLC patients. Despite significant advances in NSCLC treatment with anti-PD-(L)1 therapy, advanced disease remains largely incurable.

Melanoma remains quite prevalent with more than 96,000 new cases diagnosed in 2019 in the United States, even though the overall death rate remains low [Siegel 2019]. However, metastatic melanoma continues to retain relatively high morbidity, despite recent advances with CPI therapy. The overall survival of patients with advanced-stage melanoma has improved from −9 months before 2011 to at least 2 years and probably longer for specific mutations such as BRAF V600 mutant melanoma [Luke 2017], with a subset of patients having ongoing long-term tumor control. Melanoma is amenable to immunotherapy for various reasons, including extensive tumor infiltration by T cells, a high mutational load, and crosstalk between oncogenic signaling pathways and immunobiology. Use of ipilimumab, nivolumab and pembrolizumab has become the standard of care for advanced metastatic melanoma patients. Yet, despite long-term benefits to several patients, durable responses remain elusive. Resistance mechanisms to immunotherapies have been observed, such as loss of tumor antigen presentation and T-cell trafficking.

Head and neck squamous cell carcinoma (HNSCC) is diagnosed in approximately 65,000 patients annually in the US, and approximately 14,000 deaths occur each year secondary to HNSCC [Siegel 2019]. Globally, over 600,000 patients are diagnosed with this disease annually [Bray 2018]. Cancers of the head and neck constitute a collection of tumors that begin in the squamous cells lining the mucosal surfaces of the head and neck, referred to as HNSCC, and are categorized by the area of the head or neck in which they begin: oral cavity, pharynx (includes nasopharynx, oropharynx, and hypopharynx), paranasal sinuses, nasal cavity, and salivary glands. Infection with human papillomavirus (HPV), specifically HPV-16 and 18, are risk factors for developing HNSCC, particularly oropharyngeal cancers involving the tonsils or the base of the tongue [Chaturvedi 2011]. In the US, the incidence of oropharyngeal cancers caused by HPV infection is increasing, whereas the incidence of oropharyngeal cancers related to other causes is falling [Chaturvedi 2011].

When HNSCC is detected in its earliest stages and treated, it is curable with excellent long-term OS. However, once patients develop locally recurrent or metastatic disease, HNSCC remains essentially incurable and 5-year survival is less than 50% [ACS 2020]. Monoclonal antibodies targeting the T cell immune CPIs PD-(L)1 have recently demonstrated substantial anti-tumor activity in patients with advanced HNSCC. Pembrolizumab received accelerated approval based on response rate and durability of response in patients with recurrent or metastatic HNSCC whose disease had progressed after platinum-containing chemotherapy (2nd-line), irrespective of the HPV status. The ORR following pembrolizumab in this population is approximately 16% [Chow 2016]. Yet, although the recent advances in therapy for advanced HNSCC have increased the OS for patients, the disease remains incurable and pembrolizumab did not increase the response rate over EXTREME, just the duration of response.

Colorectal cancer (CRC) CRC is one of the most diagnosed cancers in the world, ranking second in women and third in men globally in incidence [Sung 2021]. In the USA, about 149,500 new cases of large bowel cancer are diagnosed annually, and about 52,980 Americans die from CRC, making it the third leading cause of cancer mortality for both men and women [Siegel 2021]. Approximately 50%-60% of patients afflicted with colorectal carcinoma have metastatic disease at diagnosis, with common sites of involvement including liver, lymph nodes, lung, peritoneum, and soft tissues [Van Cutsem 2006, Lee 2007, Yoo 2006], and about half of early-stage patients eventually develop metastatic or advanced disease [Martini 2017]. Survival outcomes for patients with metastatic CRC are dismal, with the 5-year survival rate approximately 14% [Bray 2018].

Compared to HNSCC, patients with CRC have had only modest improvement in survival with new therapies, and this is largely due to the lack of efficacy with anti-PD-(L)1 therapy, outside of the small group of patients with microsatellite instability high (MSI-H) CRC. The exact way in which microsatellite stable (MSS) CRC evades immune detection and destruction is yet to be fully understood. MSS CRC can be classified into 2 patterns representative of non-inflamed tumors. The first is through immunological ignorance, in which tumors are poorly infiltrated by lymphocytes; and second, through an immunosuppressive microenvironment created to evade immune surveillance by recruiting myeloid-derived suppressor cells or by secreting factors including TGFβ, to suppress chemokines and cytokines required to facilitate T-cell infiltration into tumors [Hegde 2016].

Refinements in conventional therapies such as chemotherapy, radiotherapy, surgery, and targeted therapies and recent advances in immunotherapies have improved outcomes in patients with advanced solid tumors. In the last few years, the FDA and European Medicines Agency have approved six CPIs: one monoclonal antibody targeting the CTLA-4 pathway, ipilimumab, and six antibodies targeting PD-L/PD-L1, including atezolizumab, avelumab, durvalumab, nivolumab, cemiplimab and pembrolizumab, for the treatment of patients with multiple cancer types, mainly solid tumors [Gentzler 2016; Ribas 2017]. These approvals have dramatically changed the landscape of cancer treatment. However, the majority of cancer patients, including the majority with tumors considered “sensitive’ to these agents, e.g., melanoma, NSCLC, urothelial carcinoma, kidney cancer, and others, either do not respond or become resistant to these agents [Arora 2019]. In addition, some of the most prevalent tumors, colorectal, breast cancer and prostate cancer, have proven largely refractory to checkpoint inhibition [Borcherding 2018].

Many immunotherapy treatments have demonstrated efficacy in only a selected subgroup of cancers, such as those expressing PD-L1 [Tecentriq® 2019], with microsatellite instability (MSI)/mismatch repair (MMR) deficiency or high tumor mutational burden [Luchini 2019], and even the novel targeted agents only address very small sub-populations across tumor types [Vitrakvi® 2019]. Indeed, despite considerable early success and relatively fewer side effects compared to other systemic therapies, the majority of cancer patients do not respond to CPIs or novel targeted therapies [Arora 2019].

With cures remaining scarce in patients with advanced solid tumors, there is an unmet medical need for more effective and less toxic therapies, such as combination therapies having synergistic mechanisms of action with the immune CPIs as disclosed herein.

### SUMMARY

In one aspect, the disclosed technology relates to a method of treating or inhibiting the growth of a tumor, including: (a) selecting a patient with a cancer; and (b) administering to the patient in need thereof a combination therapy including: (i) a therapeutically effective amount of an oncolytic virus; and (ii) a therapeutically effective amount of an antibody or antigen-binding fragment thereof that binds specifically to programmed death 1 (PD-1), wherein the anti-PD-1 antibody or antigen-binding fragment thereof includes three heavy chain complementarity determining regions (HCDR1, HCDR2 and HCDR3) of a heavy chain variable region (HCVR) including the amino acid sequence of SEQ ID NO: 1 and three light chain complementarity determining regions (LCDR1, LCDR2 and LCDR3) of a light chain variable region (LCVR) including the amino acid sequence of SEQ ID NO: 2.

In some embodiments, the oncolytic vesiculovirus includes an oncolytic vesicular stomatitis virus (VSV). In some embodiments, the VSV includes a recombinant VSV. In some embodiments, the recombinant VSV expresses a cytokine. In some embodiments, the cytokine includes an interferon-beta (IFNb). In some embodiments, a nucleic acid sequence encoding the IFNb is positioned between M and G vial genes. In some embodiments, the recombinant VSV further expresses a sodium/iodide symporter (NIS). In some embodiments, a nucleic acid sequence encoding the NIS is positioned between G and L viral genes. In some embodiments, the oncolytic virus is Voyager V1. In some embodiments, the anti-PD-1 antibody or antigen-binding fragment thereof includes HCDR1, HCDR2, HCDR3, LCDR1, LCDR2, and LCDR3 including the amino acid sequences of SEQ ID NOs: 3, 4, 5, 6, 7, and 8, respectively. In some embodiments, the anti-PD-1 antibody or antigen-binding fragment thereof includes HCVR including the amino acid sequence of SEQ ID NO: 1; and LCVR including the amino acid sequence of SEQ ID NO: 2. In some embodiments, the anti-PD-1 antibody or antigen-binding fragment thereof includes a heavy chain including the amino acid sequence of SEQ ID NO: 9 and a light chain including the amino acid sequence of SEQ ID NO: 10. In some embodiments, wherein the anti-PD-1 antibody or antigen-binding fragment thereof includes cemiplimab or a bioequivalent thereof.

In some embodiments, the oncolytic virus is administered to the patient in one or more doses of 10 to 10¹⁴ TCID₅₀. In some embodiments, the oncolytic virus is administered to the patient in one or more doses of 10⁹ TCID₅₀ or 10¹¹ TCID₅₀. In some embodiments, one dose of 10⁹ TCID₅₀ oncolytic virus is administered to the patient, and another dose of 10¹¹ TCID₅₀ oncolytic virus is administered to the patient. In some embodiments, the anti-PD-1 antibody or antigen-binding fragment thereof is administered to the patient in one or more doses of about 0.1 mg/kg to about 20 mg/kg of body weight of the patient. In some embodiments, the anti-PD-1 antibody or antigen-binding fragment thereof is administered to the patient in one or more doses of about 1 mg to about 1000 mg. In some embodiments, the anti-PD-1 antibody or antigen-binding fragment thereof is administered to the patient in one or more doses of 350 mg.

In some embodiments, the oncolytic virus is administered to the patient intratumorally or intravenously. In some embodiments, the anti-PD-1 antibody or antigen-binding fragment thereof administered to the patient intravenously, subcutaneously or intraperitoneally.

In some embodiments, the method further includes administering a therapeutically effective amount of a CTLA-4 inhibitor including ipilimumab or a bioequivalent thereof. In some embodiments, wherein the CTLA-4 inhibitor is ipilimumab.

In some embodiments, the method includes concurrently administering one or more doses of the oncolytic virus and one or more doses of the CTLA-4 inhibitor. In some embodiments, the method includes administering one or more doses of the oncolytic virus and one or more doses of the CTLA-4 inhibitor before administering one or more doses of the anti-PD-1 antibody or antigen-binding fragment thereof. In some embodiments, the method includes administering one or more doses of the oncolytic virus and one or more doses of the CTLA-4 inhibitor 7 days before administering one or more doses of the anti-PD-1 antibody or antigen-binding fragment thereof.

In some embodiments, the CTLA-4 inhibitor is administered to the patient in one or more doses of about 0.1 mg/kg to about 15 mg/kg of body weight of the patient. In some embodiments, the CTLA-4 inhibitor is administered to the patient in one or more doses of about 1 mg to about 600 mg. In some embodiments, the CTLA-4 inhibitor is administered to the patient in one or more doses of 50 mg. In some embodiments, the CTLA-4 inhibitor is administered to the patient intravenously, subcutaneously or intraperitoneally.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, administration of the combination therapy leads to at least one therapeutic effect selected from: reduction in tumor cell number, tumor regression, partial response, and complete response. In some embodiments, administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with a monotherapy selected from: the oncolytic virus and the anti-PD-1 antibody or antigen-binding fragment thereof. In some embodiments, administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with oncolytic virus monotherapy. In some embodiments, the tumor growth is inhibited by at least 50% as compared to an untreated patient.

In some embodiments, administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with a monotherapy selected from: the oncolytic virus, the anti-PD-1 antibody or antigen-binding fragment thereof, and the CTLA-4 inhibitor. In some embodiments, the tumor growth is inhibited by at least 50% as compared to a patient administered the oncolytic virus, the anti-PD-1 antibody or antigen-binding fragment thereof, or the CTLA-4 inhibitor as monotherapy. In some embodiments, the tumor growth is inhibited by at least 50% as compared to a patient administered any two of the oncolytic virus, the anti-PD-1 antibody or antigen-binding fragment thereof, and the CTLA-4 inhibitor. In some embodiments, the method further includes administering an additional therapeutic agent or therapy to the patient. In some embodiments, the additional therapeutic agent or therapy is selected from: radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFP) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an anti-inflammatory drug, and combinations thereof.

In another aspect, the disclosed technology relates to a combination of an oncolytic virus, an antibody or antigen-binding fragment thereof that binds specifically to programmed death 1 (PD-1), and a CTLA-4 inhibitor including ipilimumab or a bioequivalent thereof, wherein the combination is for use in a method of treating or inhibiting the growth of a tumor, the method including: (a) selecting a patient with a cancer; and (b) administering to the patient in need thereof a combination therapy including: (i) a therapeutically effective amount of an oncolytic virus; and (ii) a therapeutically effective amount of an antibody or antigen-binding fragment thereof that binds specifically to programmed death 1 (PD-1), wherein the anti-PD-1 antibody or antigen-binding fragment thereof includes three heavy chain complementarity determining regions (HCDR1, HCDR2 and HCDR3) of a heavy chain variable region (HCVR) including the amino acid sequence of SEQ ID NO: 1 and three light chain complementarity determining regions (LCDR1, LCDR2 and LCDR3) of a light chain variable region (LCVR) including the amino acid sequence of SEQ ID NO: 2.

In another aspect, the disclosed technology relates to a combination of an oncolytic virus, an antibody or antigen-binding fragment thereof that binds specifically to programmed death 1 (PD-1), and a CTLA-4 inhibitor including ipilimumab or a bioequivalent thereof, wherein the combination is for use in a method of treating or inhibiting the growth of a tumor, the method including: (a) selecting a patient with a cancer; and (b) administering to the patient in need thereof a combination therapy including: (i) a therapeutically effective amount of an oncolytic virus; (ii) a therapeutically effective amount of an antibody or antigen-binding fragment thereof that binds specifically to programmed death 1 (PD-1), wherein the anti-PD-1 antibody or antigen-binding fragment thereof includes three heavy chain complementarity determining regions (HCDR1, HCDR2 and HCDR3) of a heavy chain variable region (HCVR) including the amino acid sequence of SEQ ID NO: 1 and three light chain complementarity determining regions (LCDR1, LCDR2 and LCDR3) of a light chain variable region (LCVR) including the amino acid sequence of SEQ ID NO: 2; and (iii) a therapeutically effective amount of a CTLA-4 inhibitor including ipilimumab or a bioequivalent thereof.

In another aspect, the disclosed technology relates to a kit including an oncolytic virus and an antibody or antigen-binding fragment thereof that binds specifically to programmed death 1 (PD-1), in combination with written instructions for use of a therapeutically effective amount of a combination of the oncolytic virus and the anti-PD-1 antibody or antigen-binding fragment thereof for treating or inhibiting the growth of a tumor of a patient.

In another aspect, the disclosed technology relates to a kit including an oncolytic virus, an antibody or antigen-binding fragment thereof that binds specifically to programmed death 1 (PD-1), and a CTLA-4 inhibitor including ipilimumab or a bioequivalent thereof, in combination with written instructions for use of a therapeutically effective amount of a combination of the oncolytic virus, the anti-PD-1 antibody or antigen-binding fragment thereof, and the CTLA-4 inhibitor for treating or inhibiting the growth of a tumor of a patient.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a schematic of the study design described in Example 1 herein. Abbreviations: 1 L=first line; 2L-second line; CPI=checkpoint inhibitor; DRC=data review committee; IV=intravenous; NSCLC=non-small cell lung cancer; ORR=overall response rate; PD-(L)1=programmed death-(ligand)1; TCID₅₀=median tissue culture infectious dose; VV1=Voyager V1.

FIG. 2 is a schematic of the study design described in Example 2 herein.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

The present disclosure generally relates to novel double or triple combination therapies of an oncolytic virus, a PD-1 pathway inhibitor, and a CTLA-4 inhibitor that exhibit superior activity in inhibiting tumor growth as compared to any of the monotherapies or, for the triple combinations, as compared to any of the dual combination therapies of the oncolytic virus, PD-1 pathway inhibitor, and CTLA-4 inhibitor.

In one aspect, this disclosure relates to the use of double or triple combination therapies in a method of treating or inhibiting the growth of a tumor, including: selecting a patient with cancer; and administering to the patient in need thereof: (i) a therapeutically effective amount of an oncolytic virus in combination with (ii) a therapeutically effective amount of a PD-1 pathway inhibitor (e.g., cemiplimab or a bioequivalent thereof) and/or (iii) a therapeutically effective amount of a CTLA-4 inhibitor (e.g., ipilimumab or a bioequivalent thereof).

As used herein, the term “patient” may be interchangeably used with the term “subject.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

As used herein, the terms “treating,” “treat,” or the like mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the patient.

In many embodiments, the terms “tumor,” “lesion,” “tumor lesion,” “cancer,” and “malignancy” are used interchangeably and refer to one or more cancerous growths. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

According to some embodiments, the present disclosure includes methods for treating, delaying, or inhibiting the growth of a tumor. In some embodiments, the present disclosure includes methods to promote tumor regression. In some embodiments, the present disclosure includes methods to reduce tumor cell load or to reduce tumor burden. In some embodiments, the present disclosure includes methods to prevent tumor recurrence.

In some embodiments, the methods comprise administering to the patient one or more doses of an oncolytic virus before, after or concurrently with administering to the patient one or more doses of a PD-1 pathway inhibitor and/or one or more doses of a CTLA-4 inhibitor. In some embodiments, one or more doses of the PD-1 pathway inhibitor can be administered in combination with one or more doses of the CTLA-4 inhibitor.

As used herein, the term “in combination with” includes sequential or concurrent administration of two or more of the oncolytic virus, PD-1 pathway inhibitor (e.g., anti-PD-1 antibody or antigen-binding fragment thereof), and CTLA-4 inhibitor (e.g., anti-CTLA-4 antibody or antigen-binding fragment thereof). In some embodiments, one therapy disclosed herein may be administered to the patient before or after the concurrent administration of the other two therapies disclosed herein.

For example, when a first therapy is administered “before” a second and/or third therapy, one or more doses of the first therapy may be administered about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 7 days, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to administering one or more doses of the second and/or third therapy.

For example, when a first therapy is administered “after” a second and/or third therapy, one or more doses of the first therapy may be administered about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 7 days, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes after administering one or more doses of the second and/or third therapy.

As used herein, “concurrent” administration means that two or more of the oncolytic virus, PD-1 pathway inhibitor (e.g., anti-PD-1 antibody or antigen-binding fragment thereof), and CTLA-4 inhibitor (e.g., anti-CTLA-4 antibody or antigen-binding fragment thereof). are administered to the patient in separate dosage forms on the same day (e.g., within a 10 minute period of time), or are administered to the patient in a single combined dosage formulation comprising two or more of the oncolytic virus, PD-1 pathway inhibitor, and CTLA-4 inhibitor.

In some embodiments, the disclosed methods may further include administering an anti-tumor therapy. Anti-tumor therapies include, but are not limited to, conventional anti-tumor therapies such as chemotherapy, radiation, surgery, or as elsewhere described herein.

In some embodiments, the treatment produces a therapeutic effect selected from one or more of: delay in tumor growth, reduction in tumor cell number, tumor regression, increase in survival, partial response, and complete response. In some embodiments, the tumor growth in the patient is delayed by at least 10 days as compared to tumor growth in an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to a patient administered the oncolytic virus, the PD-1 pathway inhibitor, or the CTLA-4 inhibitor as monotherapy. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to a patient administered the oncolytic virus alone, or only two of the oncolytic virus, PD-1 pathway inhibitor, and CTLA-4 inhibitor.

In some embodiments, administration of the combination therapy (oncolytic virus and PD-1 pathway inhibitor and/or CTLA-4 inhibitor) leads to at least one therapeutic effect selected from: reduction in tumor cell number, tumor regression, partial response, and complete response. In some embodiments, administration of the combination therapy (oncolytic virus and PD-1 pathway inhibitor and/or CTLA-4 inhibitor) leads to at least one improvement selected from: abscopal effect, delay in tumor growth, increase in overall survival, increase in progression free survival, increase in overall response rate, increase in complete response, increase in partial response, and increase in stable disease, as compared to patients treated with the oncolytic virus as monotherapy, or as compared to patients treated with the PD-1 pathway inhibitor as monotherapy, and/or as compared to patients treated with the CTLA-4 inhibitor as monotherapy.

In some embodiments, administration of the combination therapy (oncolytic virus in combination with PD-1 pathway inhibitor and/or CTLA-4 inhibitor) includes selecting a subject with a first tumor lesion and a second tumor lesion, and administering one or more doses of the combination therapy to the first tumor lesion but not the second tumor lesion wherein the administration leads to tumor regression in both the first and second (i.e., the administered and unadministered) tumor lesions, thereby achieving an abscopal effect.

### Oncolytic Viruses

Oncolytic viruses are cancer therapies that employ engineered or naturally evolved viruses of cancer tropism to incite tumor cell death in the treated patient. In general, when a replicating oncolytic virus is administered, infected tumor cells have the potential to produce progeny virus, allowing destructive infection to spread to neighboring tumor cells. The potential for viral replication is determined by the cell's ability to sense and respond to the viral infection.

In some embodiments, the oncolytic virus is a recombinant VSV (e.g., VV1). In some embodiments, the oncolytic virus is a replication competent oncolytic rhabdovirus. Such oncolytic rhabdoviruses include, without limitation, wild type or genetically modified Arajas virus, Chandipura virus, Cocal virus, Isfahan virus, Maraba virus, Piry virus, Vesicular stomatitis Alagoas virus, Vesicular stomatitis virus (VSV), BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande ci chlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus.

Vesicular stomatitis virus (VSV), as indicated above, is a member of the Rhabdoviridae family. The VSV genome is a single molecule of negative-sense RNA that encodes 5 major polypeptides: a nucleocapsid (N) polypeptide, a phosphoprotein (P) polypeptide, a matrix (M) polypeptide, a glycoprotein (G) polypeptide, and a viral polymerase (L) polypeptide.

In some embodiments, the oncolytic virus is a wild type or recombinant VSV. In some embodiments, the recombinant VSV comprises one or more mutations, such as an M51R substitution (also herein referred to as VSV-M51R).

In some embodiments, the oncolytic virus may be engineered to express one or more cytokines, such as interferon-beta (IFNb). In some embodiments, IFNb (e.g., interferon beta-1a) can be a human or mouse IFNb or a variant thereof. In some embodiments, IFNb comprises an amino acid sequence having at least 90% (e.g., 90%, 95%, 96%, 97%, 98%, 99%) sequence identity to the amino acid sequence of SEQ ID NO: 23 or 24, or comprises the amino acid sequence of SEQ ID NO: 23 or 24. In some embodiments, a nucleic acid sequence encoding the IFNb is positioned between M and G viral genes. Such a position allows the virus to express an amount of IFNb polypeptide that is effective to activate anti-viral immune responses in non-cancerous tissue, and thus alleviate potential viral toxicity without impeding efficient viral replication in cancer cells.

In some embodiments, the recombinant VSV further expresses a sodium/iodide symporter (NIS) or a variant thereof. In some embodiments, the NIS comprises an amino acid sequence having at least 90% (e.g., 90%, 95%, 96%, 97%, 98%, 99%) sequence identity to the amino acid sequence of SEQ ID NO: 25 or comprises the amino acid sequence of SEQ ID NO: 25. In some embodiments, a nucleic acid sequence encoding the NIS is positioned between G and L viral genes which allows appropriate expression levels of NIS polypeptides.

In certain embodiments, the oncolytic virus is a recombinant VSV known in the art as Voyager V1 described in, e.g., U.S. Pat. No. 9,428,736, which is hereby incorporated by reference in its entirety.

### PD-1 Pathway Inhibitors

In some embodiments, the methods disclosed herein include administering a therapeutically effective amount of a PD-1 pathway inhibitor. As used herein, a “PD-1 pathway inhibitor” refers to any molecule capable of inhibiting, blocking, abrogating or interfering with the activity or expression of PD-1. In some embodiments, the PD-1 pathway inhibitor can be an antibody, a small molecule compound, a nucleic acid, a polypeptide, or a functional fragment or variant thereof. Non-limiting examples of suitable PD-1 pathway inhibitors include anti-PD-1 antibodies and antigen-binding fragments thereof, anti-PD-L1 antibodies and antigen-binding fragments thereof, and anti-PD-L2 antibodies and antigen-binding fragments thereof.

Other non-limiting examples of suitable PD-1 pathway inhibitors include RNAi molecules such as anti-PD-1 RNAi molecules, anti-PD-L1 RNAi, and anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 pathway inhibitors are described in, e.g., U.S. Pat. No. 9,308,236, U.S. Ser. No. 10/011,656, and US 20170290808, the portions of which that identify PD-1 pathway inhibitors are hereby incorporated by reference.

The term “antibody,” as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain comprises a heavy chain variable region (“HCVR” or “VH”) and a heavy chain constant region (comprised of domains CH1, CH2, and CH3). Each light chain comprises a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L}domain, the V_{H} and V_{L} domains may be situated relative to one another in any suitable arrangement. For example, the variable region may be dimeric and contain V_{H}-V_{H}, V_{H}-V_{L} or V_{L}—V_{L} dimers. Alternatively, the antigen-binding fragment of an antibody may contain a monomeric V_{H} or V_{L} domain.

In some embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (Xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In some embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Anti-PD-1 Antibodies and Antigen-Binding Fragments Thereof

In some embodiments, PD-1 pathway inhibitors used in the methods disclosed herein are antibodies or antigen-binding fragments thereof that specifically bind PD-1 (e.g., anti-PD-1 antibodies). The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments, the anti-PD-1 antibody, or antigen-binding fragment thereof comprises a heavy chain variable region (HCVR), light chain variable region (LCVR), and/or complementarity determining regions (CDRs) comprising the amino acid sequences of any of the anti-PD-1 antibodies set forth in U.S. Pat. No. 9,987,500, which is hereby incorporated by reference in its entirety.

In certain exemplary embodiments, the anti-PD-1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2.

According to some embodiments, the anti-PD-1 antibody or antigen-binding fragment thereof comprises three HCDRs (HCDR1, HCDR2, and HCDR3) and three LCDRs (LCDR1, LCDR2, and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8.

In yet other embodiments, the anti-PD-1 antibody or antigen-binding fragment thereof comprises an HCVR comprising SEQ ID NO: 1 and an LCVR comprising SEQ ID NO: 2. In some embodiments, the antibody comprises a heavy chain comprising the amino acid sequence of SEQ ID NO: 9. In some embodiments, the anti-PD-1 antibody comprises a light chain comprising the amino acid sequence of SEQ ID NO: 10.

An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as cemiplimab (also known as REGN2810; LIBTAYO®).

According to certain exemplary embodiments, the methods of the present disclosure comprise the use of cemiplimab or a bioequivalent thereof. As used herein, the term “bioequivalent” with respect to a PD-1 pathway inhibitor refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple doses. In the context of the present disclosure, the term “bioequivalent” includes antigen-binding proteins that bind to PD-1 and do not have clinically meaningful differences with cemiplimab with respect to safety, purity and/or potency.

According to some embodiments of the present disclosure, the anti-human PD-1, or antigen-binding fragment thereof, comprises a HCVR having at least 90% (e.g., 90%, 95%, 96%, 97%, 98%, 99%) sequence identity to SEQ ID NO: 1.

According to some embodiments of the present disclosure, the anti-human PD-1, or antigen-binding fragment thereof, comprises a LCVR having (e.g., 90%, 95%, 96%, 97%, 98%, 99%) sequence identity to SEQ ID NO: 2. Sequence identity may be measured by methods known in the art (e.g., GAP, BESTFIT, and BLAST).

According to some embodiments of the present disclosure, the anti-human PD-1 or antigen-binding fragment thereof comprises a HCVR comprising an amino acid sequence of SEQ ID NO: 1 having no more than 10 amino acid substitutions. According to some embodiments of the present disclosure, the anti-human PD-1 or antigen-binding fragment thereof comprises a LCVR comprising an amino acid sequence of SEQ ID NO: 2 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

Other anti-PD-1 antibodies or antigen-binding fragments thereof that can be used in the context of the methods of the present disclosure include, e.g., the antibodies referred to and known in the art as nivolumab, pembrolizumab, MED10608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO 2006/121168, WO 2009/114335. The portions of all of the aforementioned publications that identify anti-PD-1 antibodies are hereby incorporated by reference.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the present disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Anti-PD-L1 Antibodies and Antigen-Binding Fragments Thereof

In some embodiments, PD-1 pathway inhibitors used in the methods disclosed herein are antibodies or antigen-binding fragments thereof that specifically bind PD-L1 (e.g., anti-PD-L1 antibodies). For example, an antibody that “specifically binds” PD-L1, as used in the context of the present disclosure, includes antibodies that bind PD-L1 or a portion thereof with a Ko of about 1×10⁻⁸ M or less (e.g., a smaller Ko denotes a tighter binding). A “high affinity” anti-PD-L1 antibody refers to those mAbs having a binding affinity to PD-L1, expressed as Ko of at least 10⁻⁸ M, such as 10⁻⁹ M, 10⁻¹⁰ M, 10⁻¹¹ M, or 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

According to certain exemplary embodiments, the anti-PD-L1 antibody or antigen-binding fragment thereof comprises a heavy chain variable region (HCVR), light chain variable region (LCVR), and/or complementarity determining regions (CDRs) comprising the amino acid sequences of any of the anti-PD-L1 antibodies set forth in U.S. Pat. No. 9,938,345, which is hereby incorporated by reference in its entirety.

In certain exemplary embodiments, an anti-PD-L1 antibody or antigen-binding fragment thereof that can be used in the context of the present disclosure comprises the heavy chain complementarity determining regions (HCDRs) of a heavy chain variable region (HCVR) comprising SEQ ID NO: 11 and the light chain complementarity determining regions (LCDRs) of a light chain variable region (LCVR) comprising SEQ ID NO: 12. An exemplary anti-PD-L1 antibody comprising a HCVR of SEQ ID NO: 11 and a LCVR of SEQ ID NO: 12 is REGN3504.

According to some embodiments of the present disclosure, the anti-human PD-L1 antibody, or antigen-binding fragment thereof, comprises a HCVR having at least 90% (e.g., 90%, 95%, 96%, 97%, 98%, 99%) sequence identity to SEQ ID NO: 11. According to some embodiments of the present disclosure, the anti-human PD-L1 antibody, or antigen-binding fragment thereof, comprises a LCVR having at least 90% (e.g., 90%, 95%, 96%, 97%, 98%, 99%) sequence identity to SEQ ID NO: 12.

According to some embodiments of the present disclosure, the anti-human PD-L1 antibody, or antigen-binding fragment thereof, comprises a HCVR comprising an amino acid sequence of SEQ ID NO: 11 having no more than 10 amino acid substitutions. According to some embodiments of the present disclosure, the anti-human PD-L1 antibody, or antigen-binding fragment thereof, comprises a LCVR comprising an amino acid sequence of SEQ ID NO: 12 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-L1 antibodies that can be used in the context of the methods of the present disclosure include, e.g., the antibodies referred to and known in the art as MDX-1105, atezolizumab (TECENTRIQ™), durvalumab (IMFINZI™), avelumab (BAVENCIO™) LY3300054, FAZ053, STI-1014, CX-072, KN035 (Zhang et al., Cell Discovery, 3,170004 (March 2017)), CK-301 (Gorelik et al., American Association for Cancer Research Annual Meeting (AACR), 2016 Apr. 4 Abstract 4606), or any of the other anti-PD-L1 antibodies set forth in U.S. Pat. Nos. 7,943,743, 8,217,149, 9,402,899, 9,624,298, and 9,938,345, and in patent publications WO 2007/005874, WO 2010/077634, WO 2013/181452, WO 2013/181634, WO 2016/149201, WO 2017/034916, or EP3177649. The portions of all of the aforementioned publications that identify anti-PD-L1 antibodies are hereby incorporated by reference.

### Anti-PD-L2 Antibodies and Antigen-Binding Fragments Thereof

In some embodiments, PD-1 pathway inhibitors used in the methods disclosed herein are antibodies or antigen-binding fragments thereof that specifically bind PD-L2 (e.g., anti-PD-L2 antibodies). For example, an antibody that “specifically binds” PD-L2, as used in the context of the present disclosure, includes antibodies that bind PD-L2 or a portion thereof with a KD of about 1×10⁻⁸ M or less (e.g., a smaller KD denotes a tighter binding). A “high affinity” anti-PD-L2 antibody refers to those mAbs having a binding affinity to PD-L2, expressed as KD of at least 10⁻⁸ M, such as 10⁻⁹ M, 10⁻¹⁰ M, 10⁻¹¹ M, or 10⁻¹² M, as measured by surface plasmon resonance, e.g., BIACORE™ or solution-affinity ELISA. An isolated antibody that specifically binds human PD-L2 may, however, have cross-reactivity to other antigens, such as PD-L2 molecules from other (non-human) species.

Anti-PD-L2 antibodies that can be used in the context of the methods of the present disclosure include, e.g., the anti-PD-L2 antibodies set forth in U.S. Pat. Nos. 8,552,154 and 10,647,771. The portions of all of the aforementioned publications that identify anti-PD-L2 antibodies are hereby incorporated by reference.

### CTLA-4 Inhibitors

In some embodiments, the methods disclosed herein include administering a therapeutically effective amount of a CTLA-4 inhibitor. As used herein, a “CTLA-4 inhibitor” refers to any molecule capable of inhibiting, blocking, abrogating or interfering with the activity or expression of CTLA-4. In some embodiments, the CTLA-4 inhibitor can be an antibody, a small molecule compound, a nucleic acid, a polypeptide, or a functional fragment or variant thereof. Non-limiting examples of suitable CTLA-4 inhibitors include anti-CTLA-4 antibodies and antigen-binding fragments thereof. Other non-limiting examples of suitable CTLA-4 inhibitors include RNAi molecules such as anti-CTLA-4 RNAi molecules and dominant negative proteins such as a dominant negative CTLA-4 protein.

### Anti-CTLA-4 Antibodies and Antigen-Binding Fragments Thereof

In some embodiments, CTLA-4 inhibitors used in the methods disclosed herein are antibodies or antigen-binding fragments thereof that specifically bind CTLA-4 (e.g., anti-CTLA-4 antibodies). The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CTLA-4, as used in the context of the present disclosure, includes antibodies that bind CTLA-4 antibody or a portion thereof with a Ko of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CTLA-4 may, however, have cross-reactivity to other antigens, such as CTLA-4 molecules from other (non-human) species.

In certain exemplary embodiments, the anti-CTLA-4 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 13 and the light chain complementarity determining regions (LCDRs) of a light chain variable region (LCVR) comprising the amino acid sequence of SEQ ID NO: 14.

According to some embodiments, the anti-CTLA-4 antibody or antigen-binding fragment thereof comprises three HCDRs (HCDR1, HCDR2, and HCDR3) and three LCDRs (LCDR1, LCDR2, and LCDR3), wherein the HCDR1 comprises the amino acid sequence of SEQ ID NO: 15; the HCDR2 comprises the amino acid sequence of SEQ ID NO: 16; the HCDR3 comprises the amino acid sequence of SEQ ID NO: 17; the LCDR1 comprises the amino acid sequence of SEQ ID NO: 18; the LCDR2 comprises the amino acid sequence of SEQ ID NO: 19; and the LCDR3 comprises the amino acid sequence of SEQ ID NO: 20.

In yet other embodiments, the anti-CTLA-4 antibody or antigen-binding fragment thereof comprises an HCVR comprising the amino acid sequence of SEQ ID NO: 13 and an LCVR comprising the amino acid sequence of SEQ ID NO: 14. In some embodiments, the antibody comprises a heavy chain comprising the amino acid sequence of SEQ ID NO: 21. In some embodiments, the anti-CTLA-4 antibody comprises a light chain comprising the amino acid sequence of SEQ ID NO: 22.

An exemplary antibody comprising a heavy chain variable region comprising the amino acid sequence of SEQ ID NO: 13 and a light chain variable region comprising the amino acid sequence of SEQ ID NO: 14 is the fully human anti-CTLA-4 antibody known as REGN4659.

According to certain exemplary embodiments, the methods of the present disclosure comprise the use of REGN4659 or a bioequivalent thereof. As used herein, the term “bioequivalent” with respect to a CTLA-4 inhibitor refers to anti-CTLA-4 antibodies or CTLA-4-binding proteins or fragments thereof that are pharmaceutical equivalents or pharmaceutical alternatives whose rate and/or extent of absorption do not show a significant difference with that of a reference antibody (e.g., REGN4659) when administered at the same molar dose under similar experimental conditions, either single dose or multiple doses. In the context of the present disclosure, the term “bioequivalent” includes antigen-binding proteins that bind to CTLA-4 and do not have clinically meaningful differences with REGN4659 with respect to safety, purity and/or potency.

According to some embodiments of the present disclosure, the anti-human CTLA-4, or antigen-binding fragment thereof, comprises a HCVR having at least 90% (e.g., 90%, 95%, 96%, 97%, 98%, 99%) sequence identity to the amino acid sequence of SEQ ID NO: 13.

According to some embodiments of the present disclosure, the anti-human CTLA-4, or antigen-binding fragment thereof, comprises a LCVR having (e.g., 90%, 95%, 96%, 97%, 98%, 99%) sequence identity to the amino acid sequence of SEQ ID NO: 14.

According to some embodiments of the present disclosure, the anti-human CTLA-4, or antigen-binding fragment thereof, comprises a HCVR comprising an amino acid sequence of SEQ ID NO: 13 having no more than 10 amino acid substitutions. According to some embodiments of the present disclosure, the anti-human CTLA-4, or antigen-binding fragment thereof, comprises a LCVR comprising an amino acid sequence of SEQ ID NO: 14 having no more than 10 amino acid substitutions.

Also within the scope of this disclosure are variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-CTLA-4 antibodies or antigen-binding fragments thereof that can be used in the context of the methods of the present disclosure include, e.g., the antibodies referred to and known in the art as ipilimumab, tremelimumab, or any of the anti-CTLA-4 antibodies set forth in U.S. Pat. Nos. 7,527,969, 8,779,098, 7,666,424, 7,737,258, 7,740,845, 8,148,154, 8,414,892, 8,501,471, and 9,062,110; and in patent publications US2013/0078234, US2010/0143245, WO2017062615A2, WO 2004/001381, and WO 2012/147713. The portions of all of the aforementioned publications that identify anti-CTLA-4 antibodies are hereby incorporated by reference.

### Pharmaceutical Compositions and Administration

The present disclosure includes methods which comprise administering an oncolytic virus, a PD-1 pathway inhibitor, and/or a CTLA-4 inhibitor to a subject wherein the antibody or antibodies are contained within a separate or combined (single) pharmaceutical composition. The pharmaceutical compositions of this disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water, and water-in-oil emulsions, emulsions carbowax (polyethylene glycols of various molecular weights), semi-solid gels, and semi-solid mixtures containing carbowax. See also Powell et al. PDA (1998) J Pharm Sci Technol 52:238-311.

Various delivery systems are known and can be used to administer the pharmaceutical composition of the present disclosure, e.g., encapsulation in liposomes, microparticles, microcapsules, recombinant cells capable of expressing the mutant viruses, receptor-mediated endocytosis (see, e.g., Wu et al., 1987, J. Biol. Chem. 262: 4429-4432). Methods of administration include, but are not limited to, intradermal, intramuscular, intratumoral, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents.

A pharmaceutical composition comprising an oncolytic virus, a PD-1 pathway inhibitor, or a CTLA-4 inhibitor can be delivered intratumorally, subcutaneously or intravenously with a standard needle and syringe. In some embodiments, one or more of the oncolytic virus, PD-1 pathway inhibitor and/or CTLA-4 inhibitor may be delivered by more than one route of administration—e.g., one or more doses administered intratumorally and one or more doses administered intravenously, or by some other route of administration. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered, and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, e.g., Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure also provides kits comprising an oncolytic virus and a PD-1 pathway inhibitor, in combination with written instructions for use of a therapeutically effective amount of a combination of the oncolytic virus and the PD-1 pathway inhibitor for treating or inhibiting the growth of a tumor of a patient.

The present disclosure also provides kits comprising an oncolytic virus and a CTLA-4 inhibitor, in combination with written instructions for use of a therapeutically effective amount of a combination of the oncolytic virus and the CTLA-4 inhibitor for treating or inhibiting the growth of a tumor of a patient.

The present disclosure also provides kits comprising an oncolytic virus, a PD-1 pathway inhibitor, and a CTLA-4 inhibitor, in combination with written instructions for use of a therapeutically effective amount of a combination of the oncolytic virus, the PD-1 pathway inhibitor, and the CTLA-4 inhibitor for treating or inhibiting the growth of a tumor of a patient.

### Administration Regimens

The methods of the present disclosure may include administering to a subject an oncolytic virus, a PD-1 pathway inhibitor (e.g., an anti-PD-1, anti-PD-L1, or anti-PD-L2 antibody, or antigen-binding fragment thereof), or a CTLA-4 inhibitor (e.g., anti-CTLA-4 antibody or antigen-binding fragment thereof) at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. The methods of the present disclosure may also include administering a single dose each of an oncolytic virus, a PD-1 pathway inhibitor, or a CTLA-4 inhibitor.

In some embodiments, at least one of the oncolytic virus, the PD-1 pathway inhibitor, or the CTLA-4 inhibitor is administered to the patient once a day, once every two days, once every three days, once every four days, once every five days, once every week, once every two weeks, or once every three weeks.

In some embodiments, the oncolytic virus, the PD-1 pathway inhibitor, and the CTLA-4 inhibitor are administered concurrently to the patient.

In some embodiments, the methods may include sequentially administering to the subject two or more of the oncolytic virus, the PD-1 pathway inhibitor, and the CTLA-4 inhibitor. In some embodiments, the oncolytic virus is administered to the patient before or after the PD-1 pathway inhibitor and the CTLA-4 inhibitor. In some embodiments, the PD-1 pathway inhibitor is administered to the patient before or after the oncolytic virus and the CTLA-4 inhibitor. In some embodiments, the CTLA-4 inhibitor is administered to the patient before or after the oncolytic virus and the PD-1 pathway inhibitor.

In some embodiments, the methods may include sequentially administering to the subject the oncolytic virus and the PD-1 pathway inhibitor (or the CTLA-4 inhibitor). In some embodiments, the oncolytic virus is administered to the patient before or after the PD-1 pathway inhibitor. In some embodiments, the PD-1 pathway inhibitor is administered to the patient before or after the oncolytic.

As used herein, “sequentially administering” means that each dose of the oncolytic virus, the PD-1 pathway inhibitor, or the CTLA-4 inhibitor is administered to the subject at a different point in time, e.g., on different days separated by a predetermined interval (e.g., hours, days, weeks or months). The present disclosure includes methods which comprise sequentially administering to the patient a single initial dose of the oncolytic virus, the PD-1 pathway inhibitor, or the CTLA-4 inhibitor, followed by one or more secondary doses of the oncolytic virus, the PD-1 pathway inhibitor, or the CTLA-4 inhibitor, and optionally followed by one or more tertiary doses of the oncolytic virus, the PD-1 pathway inhibitor, or the CTLA-4 inhibitor. In some embodiments, the methods further comprise sequentially administering to the patient a single initial dose of the oncolytic virus, the PD-1 pathway inhibitor, or the CTLA-4 inhibitor, followed by one or more secondary doses of the oncolytic virus, the PD-1 pathway inhibitor, or the CTLA-4 inhibitor, and optionally followed by one or more tertiary doses the oncolytic virus, the PD-1 pathway inhibitor, or the CTLA-4 inhibitor.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the oncolytic virus, the PD-1 pathway inhibitor, or the CTLA-4 inhibitor. In some embodiments, however, the amount contained in the initial, secondary, and/or tertiary doses varies from one another (e.g., adjusted up or down as appropriate) during the course of treatment. In some embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, a dose of the oncolytic virus, the PD-1 pathway inhibitor, or the CTLA-4 inhibitor, which is administered to a patient prior to the administration of the very next dose in the sequence with no intervening doses.

In some embodiments, the methods may include administering to a patient any number of secondary and/or tertiary doses of the oncolytic virus, the PD-1 pathway inhibitor (e.g., anti-PD-1 antibody or antigen-binding fragment thereof), and/or the CTLA-4 inhibitor (e.g., anti-CTLA-4 antibody or antigen-binding fragment thereof). For example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, one or more doses of the oncolytic virus, the PD-1 pathway inhibitor, and/or the CTLA-4 inhibitor are administered in one or more cycles. An “initial treatment cycle” is a treatment cycle that is administered at the beginning of the treatment regimen; a “secondary treatment cycle” is a treatment cycle administered after the initial treatment cycle; and a “tertiary treatment cycle” is a treatment cycle administered after the secondary treatment cycle. In the context of the present disclosure, treatment cycles may be the same or different from each other. In some embodiments, any of the foregoing treatment cycles may have a duration of 1 week (7 days), 2 weeks (14 days), 3 weeks (21 days), 4 weeks (28 days), 5 weeks (35 days), 6 weeks (42 days), 7 weeks (49 days), 8 weeks (56 days), or more.

In some embodiments, one or more doses of the oncolytic virus, the PD-1 pathway inhibitor, and/or the CTLA-4 inhibitor are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

In some embodiments, the administration regimen for the combination includes concurrently administering the oncolytic virus (e.g., a recombinant VSV, such as VV1) and optionally the CTLA-4 inhibitor (e.g., an anti-CTLA-4 antibody or antigen-binding fragment thereof, such as ipilimumab or a bioequivalent thereof), and then subsequently administering the PD-1 pathway inhibitor (e.g., an anti-PD-1 antibody or antigen-binding fragment thereof, such as cemiplimab or a bioequivalent thereof). For example, the administration regimen for the combination may include administering the oncolytic virus and optionally the CTLA-4 inhibitor on Day 1, and then administering the PD-1 pathway inhibitor 1, 2, 3, 4, 5, 6, 7, 8, 9, 10, 11, 12, 13, or 14 days later.

In some embodiments, the administration regimen for the combination includes one or more of: intratumorally administering the oncolytic virus (e.g., a recombinant VSV, such as VV1), intravenously administering the oncolytic virus (e.g., a recombinant VSV, such as VV1), intravenously administering the CTLA-4 inhibitor (e.g., an anti-CTLA-4 antibody or antigen-binding fragment thereof, such as ipilimumab or a bioequivalent thereof), and intravenously administering the PD-1 pathway inhibitor (e.g., an anti-PD-1 antibody or antigen-binding fragment thereof, such as cemiplimab or a bioequivalent thereof).

For example, the administration regimen may include intratumorally administering a therapeutically effective amount of the oncolytic virus on the first day of a 28-day initial treatment cycle, intravenously administering a therapeutically effective amount of the oncolytic virus on the first day of the initial treatment cycle, intravenously administering a therapeutically effective amount of the CTLA-4 inhibitor on the first day of the initial treatment cycle, intravenously administering a therapeutically effective amount of the PD-1 pathway inhibitor on the eighth day of the initial treatment cycle, and intravenously administering a therapeutically effective amount of the PD-1 pathway inhibitor on the first day of the secondary and each subsequent treatment cycle.

In another example, the administration regimen may include intravenously administering a therapeutically effective amount of the PD-1 pathway inhibitor (e.g., an anti-PD-1 antibody or antigen-binding fragment thereof, such as cemiplimab or a bioequivalent thereof) on the first day of a 21-day initial treatment cycle, and intratumorally administering a therapeutically effective amount of the oncolytic virus on the first day of the initial treatment cycle.

### Dosage

The amount of the oncolytic virus, the PD-1 pathway inhibitor (e.g., an anti-PD-1 antibody or antigen-binding fragment thereof), or the CTLA-4 inhibitor (e.g., an anti-CTLA-4 antibody or antigen-binding fragment thereof) administered to a subject according to the methods disclosed herein is, generally, a therapeutically effective amount. As used herein, the term “therapeutically effective amount” means an amount of an oncolytic virus, a PD-1 pathway inhibitor, and/or a CTLA-4 inhibitor that results in one or more of: (a) a reduction in the severity or duration of a symptom or an indication of cancer, e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; (f) increase in survival of a subject with a cancer; and/or (g) a reduction in the use or need for conventional anti-cancer therapy (e.g., elimination of need for surgery or reduced or eliminated use of chemotherapeutic or cytotoxic agents) as compared to an untreated subject, a subject treated with monotherapy, or a subject treated with any two of the three therapeutic agents disclosed herein (PD-1 pathway inhibitor, CTLA-4 inhibitor and the oncolytic virus).

In some embodiments, the oncolytic virus of the combination may be administered as one or more unit doses of 10, 100, 10³, 10⁴, 10⁵, 10⁶, 10⁷, 10⁸, 10⁹, 10¹⁰, 10¹¹, 10¹², 10¹³, 10¹⁴, or more viral particles (vp) or plaque-forming units (pfu). In some embodiments, the oncolytic virus is an oncolytic rhabdovirus (e.g., wild type or genetically modified VSV) and is administered to a human with cancer as one or more dosages of 10⁴ to 10¹⁴ pfu, 10⁶ to 10¹⁴ pfu, 10⁸ to 10¹⁴ pfu, 10⁴ to 10¹² pfu, 10⁶ to 10¹² pfu, 10⁸ to 10¹² pfu, 10⁹ to 10¹¹ pfu, or 10¹⁰ to 10¹² pfu or any range(s) therebetween.

In some embodiments, the oncolytic virus of the combination may be administered as one or more unit doses of 10, 100, 10³, 10⁴, 10⁵, 10⁶, 10⁷, 10⁸, 10⁹, 10¹⁰, 10¹¹, 10¹², 10¹³, 10¹⁴, or more 50% Tissue Culture Infectious Dose (TCID₅₀). In some embodiments, the oncolytic virus is an oncolytic rhabdovirus (e.g., wild type or genetically modified VSV) and is administered to a human with cancer in one or more doses of 10⁴ to 10¹⁴ TCID₅₀, 10⁶ to 10¹⁴ TCID₅₀, 10⁸ to 10¹⁴ TCID₅₀, 10⁴ to 10¹² TCID₅₀, 10⁶ to 10¹² TCID₅₀, 10⁸ to 10¹² TCID₅₀, 10⁹ to 10¹¹ TCID₅₀, or 10¹⁰ to 10¹² TCID₅₀, or any range therebetween. In some embodiments, the oncolytic virus is a recombinant VSV, such as VV1, and is administered to a human with cancer in one or more doses of 10⁴ to 10¹⁴ TCID₅₀, 10⁶ to 10¹⁴ TCID₅₀, 10⁸ to 10¹⁴ TCID₅₀, 10⁴ to 10¹² TCID₅₀, 10⁶ to 10¹² TCID₅₀, 10⁸ to 10¹² TCID₅₀, 10⁹ to 10¹¹ TCID₅₀, 10¹⁰ to 10¹² TCID₅₀, or any range(s) therebetween. In some embodiments, a therapeutically effective amount of the oncolytic virus (e.g., a recombinant VSV, such as VV1) can be 10, 100, 10³, 10⁴, 10⁵, 10⁶, 10⁷, 10⁸, 10⁹, 10¹⁰, 10¹¹, 10¹², 10¹³, 10¹⁴, or more TCID₅₀.

In some embodiments, a therapeutically effective amount of the PD-1 pathway inhibitor (e.g., an anti-PD-1 antibody or antigen-binding fragment thereof, such as cemiplimab or a bioequivalent thereof) can be from about 0.05 mg to about 1500 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 pathway inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

The amount of a PD-1 pathway inhibitor (e.g., an anti-PD-1 antibody or antigen-binding fragment thereof) contained within an individual dose may be expressed in terms of milligrams of antibody per kilogram of subject body weight (i.e., mg/kg). In some embodiments, the PD-1 pathway inhibitor used in the methods disclosed herein may be administered to a subject at a dose of about 0.0001 to about 100 mg/kg of subject body weight. In some embodiments, an anti-PD-1 antibody may be administered at a dose of about 0.1 mg/kg to about 20 mg/kg of a patient's body weight. In some embodiments, the methods of the present disclosure comprise administration of a PD-1 pathway inhibitor (e.g., an anti-PD-1 antibody or antigen-binding fragment thereof) at a dose of about 1 mg/kg to 3 mg/kg, 1 mg/kg to 5 mg/kg, 1 mg/kg to 10 mg/kg, 3 mg/kg to 10 mg/kg, 5 mg/kg to 10 mg/kg, 1 mg/kg, 3 mg/kg, 5 mg/kg, or 10 mg/kg of a patient's body weight.

In some embodiments, each dose of the PD-1 pathway inhibitor comprises 0.1-10 mg/kg (e.g., 0.3 mg/kg, 1 mg/kg, 3 mg/kg, or 10 mg/kg) of the subject's body weight. In certain other embodiments, each dose comprises 5-1500 mg of the PD-1 pathway inhibitor (such as an anti-PD-1 antibody or antigen-binding fragment thereof), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1550 mg, 1300 mg, 1350 mg, 1400 mg, 1450 mg, or 1500 mg of the PD-1 pathway inhibitor.

In some embodiments, a therapeutically effective amount of the CTLA-4 inhibitor (e.g., an anti-CTLA-4 antibody or antigen-binding fragment thereof, such as ipilimumab or a bioequivalent thereof) can be from about 0.05 mg to about 1000 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the CTLA-4 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg.

The amount of a CTLA-4 inhibitor (e.g., an anti-CTLA-4 antibody or antigen-binding fragment thereof) contained within an individual dose may be expressed in terms of milligrams of antibody per kilogram of subject body weight (i.e., mg/kg). In some embodiments, an anti-CTLA-4 antibody may be administered at a dose of about 0.1 mg/kg to about 20 mg/kg of a patient's body weight. In some embodiments, the methods of the present disclosure comprise administration of a CTLA-4 inhibitor (e.g., an anti-CTLA-4 antibody or antigen-binding fragment thereof) at a dose of about 1 mg/kg to 3 mg/kg, 1 mg/kg to 5 mg/kg, 1 mg/kg to 10 mg/kg, 1 mg/kg, 3 mg/kg, 5 mg/kg, 10 mg/kg or 15 mg/kg of a patient's body weight.

In some embodiments, each dose of the CTLA-4 inhibitor comprises 0.1-10 mg/kg (e.g., 0.3 mg/kg, 1 mg/kg, 3 mg/kg, or 10 mg/kg) of the subject's body weight. In certain other embodiments, each dose comprises 5-1000 mg of the CTLA-4 inhibitor (such as an anti-CTLA-4 antibody or antigen-binding fragment thereof), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg of the CTLA-4 inhibitor.

In some embodiments, the methods of the present disclosure further include administering to a subject an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: radiation, surgery, a cancer vaccine, imiquimod, an anti-viral agent (e.g., cidofovir), photodynamic therapy, a lymphocyte activation gene 3 (LAG3) inhibitor (e.g., an anti-LAG3 antibody, a glucocorticoid-induced tumor necrosis factor receptor (GITR) agonist (e.g., an anti-GITR antibody), a T-cell immunoglobulin and mucin containing -3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist (e.g., a “VEGF-Trap” such as aflibercept, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)), an angiopoietin-2 (Ang2) inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor (GM-CSF), a second oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), an IL-6R inhibitor, an IL-4R inhibitor, an IL-10 inhibitor, a cytokine such as IL-2, IL-7, IL-12, IL-21, and IL-15, an antibody drug conjugate, an anti-inflammatory drug such as a corticosteroid, a non-steroidal anti-inflammatory drug (NSAID), cryotherapy, anti-HPV therapy, laser therapy, electrosurgical excision of cells with HPV, and combinations thereof.

In some embodiments, the methods further comprise administering an additional therapeutic agent, such as an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mitotane (O, P′-(DDD)), biologics (e.g., antibodies and interferons) and radioactive agents. As used herein, “a cytotoxin or cytotoxic agent” also refers to a chemotherapeutic agent and means any agent that is detrimental to cells. Examples include TAXOL (paclitaxel), temozolomide, cytochalasin B, gramicidin D, ethidium bromide, emetine, cisplatin, mitomycin, etoposide, teniposide, vincristine, vinblastine, colchicine, doxorubicin, daunorubicin, dihydroxy anthracene dione, mitoxantrone, mithramycin, actinomycin D, 1-dihydrotestosterone, glucocorticoids, procaine, tetracaine, lidocaine, propranolol, and puromycin and analogs or homologs thereof.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the term “agent” denotes a chemical compound, a mixture of chemical compounds, a biological macromolecule (such as a nucleic acid, an antibody, a protein or portion thereof, e.g., a peptide), or an extract made from biological materials, such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject. In the context of the disclosure, the term “therapeutic agent” refers to any of the PD-1 pathway inhibitors, CTLA-4 inhibitors or oncolytic viruses disclosed herein.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the present disclosure and are physiologically acceptable to the subject. Supplementary active compounds may also be incorporated into the compositions.

Doses are often expressed in relation to bodyweight. Thus, a dose which is expressed as [g, mg, or other unit]/kg (or g, mg, etc.) usually refers to [g, mg, or other unit]“per kg (or g, mg, etc.) bodyweight,” even if the term “bodyweight” is not explicitly mentioned. The treatments may include various “unit doses.” A unit dose is defined as containing a predetermined quantity of the therapeutic composition. A unit dose need not be administered as a single injection but may comprise continuous infusion over a set period of time. For oncolytic viruses, a unit dose may be described in terms of plaque-forming units (pfu) or viral particles for viral constructs. Unit doses range from 10³, 10⁴, 10⁵, 10⁶, 10⁷, 10⁸, 10⁹, 10¹⁰, 10¹¹, 10¹², 10¹³ pfu or vp and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells. Alternatively, unit doses for oncolytic viruses are represented by TCID₅₀. “TCID₅₀” refers to “tissue culture infective dose” and is defined as the dilution of a virus required to infect 50% of a given batch of inoculated cell cultures. Various methods known to one skilled in the art may be used to calculate TCID₅₀, including the Spearman-Karber method which is utilized throughout this specification. For a description of the Spearman-Karber method, see B. W. Mahy & H. 0. Kangro, Virology Methods Manual 25-46 (1996). In some embodiments, Unit doses range from 10³, 10⁴, 10⁵, 10⁶, 10⁷, 10⁸, 10⁹, 10¹⁰, 10¹¹, 10¹², 10¹³ TCID₅₀ and higher or any ranges therebetween.

As used herein, the term “disease” is intended to be generally synonymous and is used interchangeably with the terms “disorder” and “condition” (as in medical condition), in that all reflect an abnormal condition (e.g., cancer) of the human or animal body or of one of its parts that impairs normal functioning, is typically manifested by distinguishing signs and symptoms, and causes the human or animal to have a reduced duration or quality of life.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having,” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the present disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the present disclosure and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Phase 2 Trial of Voyager V1 (Vesicular Stomatitis Virus Expressing NIS and Human Interferon Beta, VV1), in Combination with Cemiplimab in Patients with Hepatocellular Carcinoma, Non-Small Cell Lung Cancer, Melanoma or Endometrial Carcinoma

This example relates to an open label, Simon 2-stage phase 2 clinical trial to determine the anti-tumor activity of Voyager V1 (VV1) in combination with cemiplimab alone and together with ipilimumab in patients with hepatocellular carcinoma, non-small cell lung cancer (NSCLC), melanoma or endometrial carcinoma.

Background: VV1 is an oncolytic vesicular stomatitis virus engineered to express human interferon beta (IFNβ) to enhance cellular anti-tumor immune responses and tumor selectivity. There is a need for novel immunotherapies to address patient populations that never responded, or no longer respond, to immune checkpoint inhibitors (CPIs). Phase 1 studies demonstrated VV1 anti-tumor activity in several malignancies with or without a CPI. This example relates to optimizing VV1 efficacy in combination with cemiplimab, an anti-PD1 antibody approved for lung, basal and squamous cell skin cancers, in two ways: use of a higher dose, and triple combination in representative patient populations.

Patients with advanced melanoma (after progression on an anti-PD1) are enrolling in this study, which will eventually include first line NSCLC patients whose disease is amenable to immunotherapy. The study is first exploring the preliminary anti-tumor activity, safety, and immunogenic activity of the combination of intravenous (IV) VV1 at a dose of 1.0×101 once on D1 (Day 1) followed by IV cemiplimab Q3W starting on D8, or the same regimen with an additional intratumoral injection of VV1 1.0×10⁹ TCID₅₀ once on D1. Patients receive IV cemiplimab Q3W until confirmed disease progression or intolerable toxicity. Once at least six patients have been safely treated across the two melanoma doublet cohorts, a 3rd (triplet) cohort will open to add a single dose of ipilimumab 50 mg on D1 in the same patient population. Once at least six melanoma patients have received the triplet safely, the first line NSCLC cohort will initiate triplet therapy. Cohorts will be expanded based on a Simon 2-stage design using a type I error rate of 0.05 and power of 85%. Null overall response rate (ORR) is 10% with a target of 35% for 2nd line melanoma and null ORR is 40% in 1st line NSCLC with a true target of 70%. Melanoma cohorts will require a response in ≥2 of 10 patients in the 1st stage to add 11 more in the 2nd stage, while NSCLC will first need 5/9 evaluable patients to respond, then an additional 13 to complete the design. Response is assessed at week 7 then Q9W per RECIST v1.1. The study includes serial biopsies in ≥3/10 patients in Stage 1 of each of the IV melanoma cohorts (doublet and triplet therapy), all patients in Stage 2 of these IV melanoma cohorts, and all patients in both Stage 1 and Stage 2 of the intravenous/intratumoral (IV/IT) melanoma cohort, to permit a thorough investigation of the impact of the three immunotherapies under investigation.

Study Rationale: Cancer immunotherapy has demonstrated significant clinical success resulting in durable responses in patients with advanced, previously refractory cancer. Specifically, immune CPI agents that block signals that tumors co-opt to dampen immune responses allowing activation of T-cell mediated anti-tumor immunity, have demonstrated clinical efficacy and are approved for the treatment of several indications including melanoma, NSCLC, urothelial, head and neck, gastric, hepatocellular carcinoma (HCC) and other tumors. A majority of patients remain resistant to CPI therapy, with response rates ranging from 10-40% depending on the indication. Populations of patients whose disease is resistant or refractory to CPIs are now emerging as a significant unmet medical need. Not all NSCLC or melanoma patients, in whom anti-PD-(L)1 therapy has become standard of care, obtain long term durable benefits from such treatment. Once resistant, these patients have limited treatment options. Additionally, patients, among these indications, who do not respond and are refractory to CPI therapy also continue to have an unmet medical need.

It is predicted that combination therapy with oncolytic VSV-IFNβ-NIS and a CPI will enhance intratumoral CD8+ T-cell infiltration and activation to enhance therapeutic response in cancer patients who are previously resistant to CPI monotherapy. The present study, conducted in a melanoma patient population that has failed single agent CPI, is designed to provide insight into putative mechanisms of re-sensitization to CPI through infusion of a single dose of oncolytic virus, with the hypothesis that cell infection will expand the available endogenous anti-tumor T cell repertoire, increase the tumor cell antigenicity for T cell recognition, enhance antigen presentation, and ultimately convert a non-inflamed or immune-excluded phenotype to a highly inflamed phenotype.

Study Objectives: The primary objective of the study is to determine the preliminary anti-tumor activity in each study cohort as measured by overall response rate (ORR) according to standard criteria (RECIST 1.1) [Eisenhauer 2009]. Secondary objectives of the study include: determining the safety and tolerability of VV1 in combination with cemiplimab; evaluating the systemic pharmacokinetics (PK) of VV1 and cemiplimab; investigating the pharmacodynamics of VV1 in the blood by measuring serum IFNβ expression; determining preliminary clinical activity of each cohort [e.g., progression-free survival (PFS), duration of response (DOR), disease control rate (DCR) and overall survival (OS)]; and evaluating the safety and efficacy of administration of VV1, cemiplimab, and ipilimumab. Other objectives include assessment of: immunocyte infiltrate in select pre- and post-treatment tumor specimens; time course of viremia, viral shedding, and virus persistence; effect on host peripheral blood immunocyte expansion/suppression; immunogenicity measured by anti-drug antibodies to cemiplimab+VV1; and ability of ruxolitinib to mitigate the risk of IFNβ toxicity.

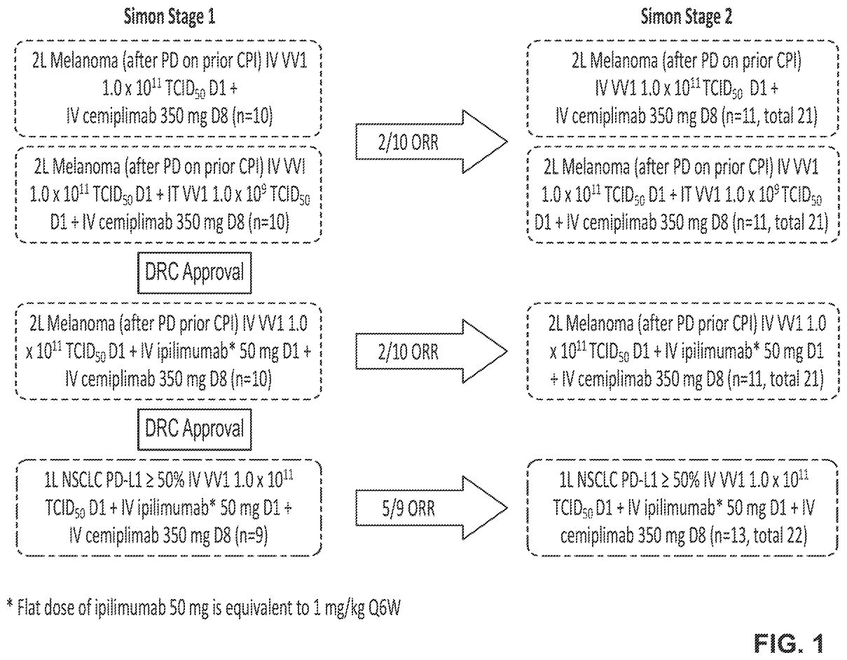

Overall Study Design: This is a Phase 2 study designed to determine the preliminary anti-tumor activity and confirm the safety of VV1 in combination with cemiplimab alone and then together with ipilimumab. The hepatocellular and endometrial cancer cohorts are closed, and the melanoma and NSCLC cohorts are modified in the following manner: VV1 dose is increased to 1.0×10¹¹ TCID₅₀ in combination with cemiplimab (doublet) and ipilimumab is subsequently added to the combination (triplet). The NSCLC cohort will enroll CPI treatment-naïve patients. The study design is illustrated in FIG. 1. Patients with advanced melanoma will be enrolled into two parallel doublet cohorts; in one cohort (IV Melanoma Cohort) patients will receive IV VV1, and patients in the other cohort (IV/IT Melanoma Cohort) will receive both IV VV1 and IT VV1; both cohorts will receive IV cemiplimab on Day 8 after receiving VV1 treatment. After completion of the high-dose doublet melanoma cohort, a subsequent triplet melanoma cohort will receive IV VV1, IV cemiplimab, and ipilimumab (1 dose) combination therapy. Finally, a cohort of patients with NSCLC will receive the triplet combination.

Doses of up to 1.7×10¹¹ TCID₅₀ VSV-IFNβ-NIS (IV monotherapy) were found to be safe and potentially more efficacious than lower doses.

All enrolled patients on this study will be required to be hospitalized overnight on Day 1 and may be discharged for outpatient follow-up on Day 2, if clinically stable. Patients are then required to undergo outpatient safety evaluations on Days 3, 4 and 5. Lab assessments performed on Days 2, 3, 4 and 5 must be evaluated and assessed for possible IFNβ toxicity before the patient is discharged to home on those days.

Patients will enroll in each melanoma doublet cohort concurrently. A safety lead-in of 3 patients in the IV cohort will be dosed and observed for 28 days and their safety data reviewed before the IT/IV melanoma cohort will enroll. A total of six (6) patients across both cohorts will be dosed and observed for one cycle. Once the combination is deemed safe, enrollment in the IV melanoma triplet cohort will ensue.

For all cohorts in the study, a Simon's two-stage design [Simon 1989] will be used. In the first stage, 10 evaluable patients will be accrued in each melanoma tumor cohort; the cohort will progress to the second stage based on a combination of safety and efficacy. If there are less than 2 objective responses in each 10-patient melanoma cohort, the cohort will be stopped; otherwise, 11 additional patients will be accrued for a total of 21 patients in each melanoma cohort. A similar design will be used for NSCLC, where the first stage will enroll 9 evaluable patients. In this NSCLC cohort a higher efficacy bar of at least 5 out of 9 patients with an objective response will be required to trigger Stage 2 for a total of 22 evaluable patients.

Patients in the IV Melanoma cohort will receive IV VV1 and patients in the V/IT Melanoma cohort will receive both IV VV1 and IT VV1 on Day 1 of Cycle 1; both cohorts will receive IV cemiplimab on Day 8 after receiving VV1 treatment. After Cycle 1 (a 28-day cycle), cemiplimab treatment will continue Q3W per the Libtayo® US Package Insert, with efficacy evaluations after Cycle 2, then every 9 weeks until progressive disease (PD). After completion of the high-dose melanoma cohort, a subsequent triplet melanoma cohort will receive IV VV1, ipilimumab (1 dose) and IV cemiplimab combination therapy. Finally, a cohort of patients with NSCLC will receive the triplet combination.

The NCI CTCAE, v5.0 will be used for grading overall toxicities and the ASTCT consensus grading will be used for grading CRS and neurologic toxicity [Lee 2019]. Safety assessments will include AEs, SAEs, PEs, vital sign measurements, ECOG status, clinical safety laboratory evaluations (hematology, serum chemistry and hepatic panels, coagulation and urinalysis) and ECGs.

Study Population: Only patients who meet all of the inclusion criteria and none of the exclusion criteria will be eligible to receive drug.

Inclusion Criteria: Patients meeting all of the following inclusion criteria at screening/Day 1 of first dosing will be eligible for participation in the study: (1) Age 18 years on day of signing informed consent. (2) Specific by tumor cohorts: (a) For the NSCLC cohort, histologically confirmed diagnosis of advanced and/or metastatic NSCLC suitable for first line immunotherapy. NSCLC harboring an activating EGFR mutation or anaplastic lymphoma kinase (ALK) rearrangement must have progressed following available EGFR or ALK targeted therapy in addition to treatment with platinum-based chemotherapy (unless ineligible for platinum therapy). (i) Able to supply archival (or fresh) formalin-fixed, paraffin-embedded tumor tissue collected within 6 months prior to enrollment for determination of programmed death ligand 1 (PD-L1) status. (ii) PD-L1 status of 50% per local standardized testing. Samples should be provided to the central lab for post-hoc centralized testing. (b) For the melanoma cohorts, histologically confirmed diagnosis of advanced and/or cutaneous metastatic melanoma in which radiological progression has been demonstrated during therapy with a PD-(L)1 immune checkpoint inhibitor and for which no existing options are considered to provide clinical benefit (only one line of PD-(L)1 therapy is permitted). Progression on ipilimumab is not required. BRAF V600 mutation patients must have progressed on, or are intolerant to, BRAF+/−MEK inhibitor therapy. i. Patients with BRAF V600-positive tumor(s) should have received prior treatment with a BRAF inhibitor (alone or in combination with a MEK inhibitor) in addition to treatment with pembrolizumab or nivolumab with or without ipilimumab or to have declined targeted therapy. NOTE: Patients with BRAF V600-positive tumors with no clinically significant tumor-related symptoms or evidence of rapidly progressive disease are not required to be treated with a BRAF inhibitor (alone or in combination with a MEK inhibitor) based on investigator's decision. For V/IT melanoma cohort: (i) At least one tumor lesion amenable to IT injection via palpation or ultrasound. Injection of deep visceral lesions is not permitted; (ii) Agrees to provide a newly obtained biopsy of injected lesions prior to start of study treatment, and to repeat biopsies twice during study treatment, and to providing the acquired tissue for biomarker analysis. Tissue obtained for the biopsy must not be previously irradiated, but a new or progressing lesion in the radiation field is acceptable. (3) For patients treated with prior anti-PD-(L)1 therapy: (a) Last dose of anti-PD-(L)1 must be within 16 weeks of initiating study treatment. (b) Patient must have received at least 4 doses on q2w, 3 doses on q3w or 2 doses on q4w schedule of the previous anti-PD-(L)1 therapy. (c) Progression on prior anti-PD-(L)1 therapy must be defined by: (i) Documented radiographic progression on a single radiographic scan if treatment with anti-PD-(L)1 was 16 weeks; (ii) Documented radiographic progression on two consecutive radiographic scans at least 4 weeks apart, if treatment with anti-PD-(L)1 therapy was between 8-16 weeks; if radiographic progression is accompanied with clinical progression, then a single scan assessment may be used; (iii) If progression was only in lymph nodes, biopsy to provide histological confirmation of progression in the lymph node is required. (4) Measurable disease based on RECIST 1.1. (5) Performance status of 0 or 1 on the ECOG Performance Scale; (6) Life expectancy of >3 months; (7) Willingness to provide biological samples required for the duration of the study including a fresh tumor biopsy sample. At least 3 out of 10 patients in the Stage 1 of each of the IV melanoma cohorts (doublet and triplet therapy) must provide pre-treatment and on-treatment fresh tumor biopsy samples. In Stage 2 of these cohorts, all patients must provide pre-treatment and on-treatment fresh tumor biopsy samples. All patients in both Stage 1 and Stage 2 of the IT/IV melanoma cohort must provide pre-treatment and on-treatment fresh tumor biopsy samples. (8) Adequate organ function defined as the laboratory values obtained 514 days prior to registration shown in Table 1; (9) Negative pregnancy test for female patients of childbearing potential; (10) Absence of active CNS involvement. NOTE: Pre-enrollment imaging of asymptomatic patients not mandatory; (11) Ability to provide written informed consent.

TABLE 1
Adequate organ function

| System | Laboratory Value |
|---|---|
| Hematological | |
| ANC | ≥1500/mcL |
| Platelets | ≥100,000/mcL |
| Hemoglobin | ≥9 g/dL |
| Renal | |
| Serum creatinine OR | ≤2.0 × ULN OR |
| Measured or calculated creatinine | ≥40 mL/min for patients with |
| clearance (CL) (per CKD-EPI) | creatinine levels > 2.0 × ULN |
| Hepatic | |
| Albumin | ≥3.0 g/dL |
| Direct bilirubin | ≤1.5 × ULN |
| AST | ≤2.5 × ULN |
| ALT | ≤2.5 × ULN |

Exclusion Criteria: Patients meeting any of the following exclusion criteria at screening/Day −1 of first dosing will not be enrolled in the study: (1) Availability of and patient acceptance of an alternative curative therapeutic option; (2) Recent or ongoing serious infection, including any active Grade 3 or higher per the NCI CTCAE, v5.0 viral, bacterial, or fungal infection within 2 weeks of registration; (3) Patients who have a diagnosis of ocular, mucosal or acral melanoma; (4) Known seropositivity for and with active infection with HIV. a. Patients who are seropositive for HIV but are receiving antiviral therapy and show non-detectable viral load and a normal CD4 T cell count for at least 6 months are eligible; (5) Seropositive for and with evidence of active viral infection with HBV. Patients who are HBsAg negative and HBV viral DNA negative are eligible. (a) Patients who had HBV but have received an antiviral treatment and show non-detectable viral DNA for 6 months are eligible; (b) Patients who are seropositive because of HBV vaccine are eligible; (6) Seropositive for and with active viral infection with HCV. a. Patients who had HCV but have received an antiviral treatment and show no detectable HCV viral RNA for 6 months are eligible; (7) Known history of active or latent TB; (8) Any concomitant serious health condition, which, in the opinion of the investigator, would place the patient at undue risk from the study, including uncontrolled hypertension and/or diabetes, clinically significant pulmonary disease (e.g., chronic obstructive pulmonary disease requiring hospitalization within 3 months) or neurological disorder (e.g., seizure disorder active within 3 months); (9) Prior therapy within the following timeframe before the planned start of study treatment as follows: (a) Small molecule inhibitors, and/or other investigational agent: s 2 weeks or 5 half-lives, whichever is shorter; (b) Chemotherapy, other monoclonal antibodies, antibody-drug conjugates, or other similar experimental therapies: 53 weeks or 5 half-lives, whichever is shorter; (c) Radioimmunoconjugates or other similar experimental therapies s 6 weeks or 5 half-lives, whichever is shorter; (10) NYHA classification Ill or IV, known symptomatic coronary artery disease, or symptoms of coronary artery disease on systems review, or known cardiac arrhythmias (atrial fibrillation or SVT) (11) Any known or suspected active organ-threatening autoimmune disease, such as inflammatory bowel disease, autoimmune hepatitis, lupus, or pneumonitis, with the exception of hypothyroidism and type 1 diabetes that are controlled with treatment; (12) Immunodeficiency or immunosuppression, including systemic corticosteroids at >10 mg/day prednisone or equivalent within 1 week prior to planned start of study treatment; (13) History of Grade 3 or 4 immune-mediated adverse reaction to immune CPIs; (14) Toxicities from previous therapies that have not resolved to a Grade 1 or less; (15) Current or historic non-infectious pneumonitis that required steroids, or current pneumonitis; (16) High volume disease, as assessed clinically by the medical monitor via parameters such as radiologic impression and tumor markers or LDH; (17) Portal vein thrombosis involving more than intrahepatic portal vein branches: thrombosis of the right or left portal vein branch or the bifurcation, partial or complete obstruction of the portal vein trunk; (18) Known concurrent malignancy. EXCEPTIONS: basal cell carcinoma of the skin, squamous cell carcinoma of the skin, or in-situ cervical cancer that has been treated with curative intent, prostate cancer confined to the prostate gland with Gleason score <6 or PSA <1, as well as any stage I cancer treated with curative intent or any prior cancer with a disease-free interval of >3 years; (19) Any other concurrent anti-cancer therapy (chemotherapy, immunotherapy, radiotherapy, or any ancillary therapy considered investigational [used for a non-FDA approved indication and in the context of a research investigation]); (20) Has received a live vaccine within 30 days of planned start of study treatment. Seasonal influenza vaccines for injection are generally inactivated flu vaccines and are allowed. COVID-19 mRNA vaccines are also allowed; intranasal influenza vaccines (e.g., Flu-Mist®) are live attenuated vaccines and are NOT allowed 4 weeks before first dose and 12 weeks after last dose of VV1 administration; (21) Any of the following because this study involves an investigational agent whose genotoxic, mutagenic and teratogenic effect on the developing fetus and newborn are unknown: (a) WOCBP*, or sexually active men, who are unwilling to practice highly effective contraception prior to the initial dose/start of the first treatment prior to the start of the first treatment, during the study, and for at least 4 weeks after the last dose. Highly effective contraceptive measures include: stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device (IUD); intrauterine hormone-releasing system (IUS); bilateral tubal ligation; vasectomized partner; and or sexual abstinence†‡. (i) Stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; (ii) IUD; IUS; (iii) Bilateral tubal ligation; (iv) Vasectomized partner (provided that the male vasectomized partner is the sole sexual partner of the WOCBP study participant and that the vasectomized partner has obtained medical assessment of surgical success for the procedure); and/or sexual abstinencet †, ‡. *WOCBP are defined as women who are fertile following menarche until becoming postmenopausal, unless permanently sterile. Permanent sterilization methods include hysterectomy, bilateral salpingectomy, and bilateral oophorectomy. A postmenopausal woman is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. Pregnancy testing and contraception are not required for women with documented hysterectomy or tubal ligation. † Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient. ‡ Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together; (22) Pregnant or nursing women; (23) Men who are unwilling to use a condom (even if they have undergone a prior vasectomy) while having vaginal intercourse, while taking the drug and for 4 weeks after stopping treatment; (24) Patients due to receive ipilimumab: a. With hypersensitivities to the excipients of ipilimumab: Hypersensitivity present for the product's excipients (Tris hydrochloride (2-amino-2-hydroxymethyl-1,3-propanediol hydrochloride), Sodium chloride, Mannitol (E421), Pentetic acid (diethylenetriaminepentaacetic acid), Polysorbate 80, Sodium hydroxide (for pH-adjustment), Hydrochloric acid (for pH-adjustment).

Study Treatments: The VSV-IFNβ-NIS as manufactured (i.e., undiluted) is in a buffer consisting of 5% sucrose, 50 mM Tris (pH 7.4), 2 mM MgCl₂. After manufacture, the virus is stored frozen at ≤−65° C. until it is prepared for use immediately prior to administration. Cemiplimab (Libtayo®) is prepared, stored and handled per its package insert. Ipilimumab (Yervoy®) is prepared, stored and handled per its package insert.

Dose Schedule and Administration: The first cycle is 28 days. Subsequent cycles are 21 days. Both IV and IT VV1 are to be administered on Day 1. Ipilimumab should also be given on Day 1 in the triplet cohorts. Cemiplimab is given Day 8 of Cycle 1 and Day 1 of Cycle 2 onwards (21-day cycles). The treatment schedule for combination therapy is shown in Table 2. Cemiplimab should be given on Day 8 of Cycle 1 (28-day cycle) and then Day 1 of each subsequent 21-day cycle for up to 2 years. Ipilimumab should be given on Day 1 in the triplet cohorts.

TABLE 2
Treatment schedule for combination

| Agent | Dose | | Route | Day |
|---|---|---|---|---|
| VV1* | 1 × 10⁹ | TCID₅₀ | IT | 1 |
| VV1 | 1.0 × 10¹¹ | TCID₅₀ | IV | 1 |
| Ipilimumab** | 50 | mg | IV | 1 |
| Cemiplimab | 350 | mg | IV | 8 of Cycle 1 (28-days), |
| | | | | then q21 days |

*Dose and schedule of IT administration only for IV/IT Melanoma Cohort.
**Ipilimumab only in triplet cohorts.

Cemiplimab administration: Cemiplimab is administered as an IV infusion over 30 minutes under close observation as per institutional standard of care. Cemiplimab should be given on Day 8 of Cycle 1 (28-day cycle) and on Day 1 of each subsequent 21-day cycle for up to 2 years.

Intravenous VVi administration: The dose of VV1 will be 1.0×10¹¹ TCID₅₀ infused IV on Day 1. The virus will be diluted in 100 mL of normal saline with 1% HSA and administered by slow IV infusion (30 minutes) under close observation.

IV/IT Melanoma cohort dose administration: For this cohort, all patients will receive both IV VV1 and IT VV1. The order of administration should be adjusted for convenience. VV1 will be administered once intratumoral (IT) at a total dose of 1×10⁹ TCID₅₀ via injection on Day 1 to one or more tumor lesions using TB syringes (or equivalent) with 20- to 23-gauge needles. The needle type can also be per the investigator's discretion, to best distribute the virus within the lesion. VV1 can be administered IT into multiple accessible lesions using an appropriate syringe and needle type for the location of the lesion(s). The rationale for dosing multiple lesions is to produce an immune response against a wider range of genetic mutations and antigenic diversity than may occur within a single lesion, which in turn is expected to reduce the chance of immune escape by the tumor.

Administration is permitted only directly into the tumor or accessible lymph node. Injection of skin, soft tissue or deep visceral lesions (e.g., liver or lung metastases) requiring radiological control via CT or MRI, or referral to an interventional radiologist is NOT permitted. If multiple lesions are designated for injection, the lesions should be at sites as disparate as possible in the body. Once lesions are identified, the maximum volume to be injected is 4 mL distributed over different injectable tumors. Lesions must be greater than 0.5 cm in size to be eligible for IT VV1 administration for a total of up to 8 lesions. Distribution of volume of infusion will be determined based on the size of lesion to be injected (Table 3):

TABLE 3
IT VV1 Administration

| Lesion size | Maximum Injection |
|---|---|
| (longest dimension) | Volume |
| ≥5.0 cm | 4.0 mL |
| >2.5 cm to 5.0 cm | 2.0 mL |
| >1.5 cm to 2.5 cm | 1.0 mL |
| >0.5 cm to 1.5 cm | 0.5 mL |

*Lesions ≤0.5 cm can only be injected if clinically responded to prior IT therapy.

IV VV1 administration will occur in a similar manner as in all other cohorts. Patients will receive a single IV dose of VSV at 1.0×10¹¹ TCID₅₀, infused over 30 minutes on Day 1. All concomitant measures mentioned above for mitigation of infusion related reactions (IRRs) should be started prior to IT administration for patients in this cohort. If the patient experiences an IRR due to the first administration of VV1, the second modality of VV1 may be postponed until Day 2-4.

Ipilimumab administration for triplet melanoma and NSCLC cohorts: All patients in triplet cohorts will receive both IV VV1 and ipilimumab on Day 1 and cemiplimab on Day 8 of Cycle 1. Ipilimumab is administered as a single 50 mg IV infusion over 90 minutes. Ipilimumab should be given on Day 1 of Cycle 1 only. The order of administration on Day 1 should be ipilimumab followed by VV1.

Treatment duration: In the absence of treatment delays due to AE(s), treatment may continue for up to a maximum of 2 years or until one of the following criteria applies: (i) disease progression; (ii) intercurrent illness that prevents further administration of treatment; (iii) AE(s) which require(s) treatment discontinuation: Any dosing interruption lasting >12 weeks with the following exceptions: (a) Dosing interruptions >12 weeks that occur for non-drug-related reasons may be allowed if approved by the PI. Prior to re-initiating treatment in a patient with a dosing interruption lasting >12 weeks, the PI must be consulted; (b) Tumor assessments should continue as per protocol even if dosing is interrupted; (iv) patient decides to withdraw from the study; (v) general or specific changes in the patient's condition render the patient unacceptable for further treatment in the judgment of the investigator. Clinical progression is defined as occurring when a patient's condition has deteriorated, and the patient cannot continue with study treatment per the investigator's judgment regardless of imaging findings; (vi) patient non-compliance; (vii) pregnancy; (viii) termination of the study by sponsor; (ix) the drug manufacturer can no longer provide the study agent; (x) if a patient has had a confirmed complete CR that has been treated for at least 24 weeks with cemiplimab and has continued at least two cycles beyond CR, discussion between the treating physician and investigator regarding discontinuation of study treatment may be considered.

Prior and concomitant illnesses and medication: Patients should receive full supportive care during the study including blood products, antibiotics and treatment of concurrent medical conditions and other newly diagnosed diseases.

All prior significant illnesses that are relevant to patient safety or that the patient has experienced prior to screening should be documented. Additional illnesses present at the time of informed consent are to be regarded as concomitant illnesses. Illnesses first occurring or detected during the study and/or worsening of a concomitant illness during the study are to be documented as AEs. If a patient enrolled in this study becomes infected with COVID-19, record any difficulties in performing protocol-specified procedures, including administering or using the investigational product or adhering to protocol-mandated visits and laboratory/diagnostic testing. The reasons for failing to obtain or for delaying assessments should be documented (e.g., identifying the specific limitation imposed by COVID-19 leading to the inability to perform the protocol-specified assessment). The COVID-19 positive test result should be collected as an AE. Imaging of patients with early stage COVID-19 pneumonia could be confused with pneumonitis, which has been described with for immune checkpoint inhibitors. COVID-19 should be entered in the differential should a pneumonitis AE be diagnosed. Once the patient has either clinically recovered from an actual COVID-19 infection or has had adequate time elapse from exposure to a high-risk situation (i.e., deemed low risk) and the patient is clinically stable, protocol-specific procedures and planned treatments may continue.

Prohibited treatment: Patients should not receive other investigational agents or participate in a device study within 3 weeks prior to study entry or receive systemic anti-cancer therapy per eligibility criteria, and during the study, and will make best efforts not to start any other investigational product or device study within 28 days after last drug administration. Enrollment in non-therapeutic studies is allowed. The following are not permitted from 1 week prior, and during study drug administration: systemic steroids >10 mg/day prednisone or equivalent unless required to treat an AE; systemic immune suppressants; other systemic anti-cancer therapy; live vaccines (should also be avoided for 3 months after last dose of VV1 administration, unless risk outweighs the benefit.)

Descriptions of assessments: Medical history will be recorded during screening to ensure eligibility of the patients and will focus on relevant current or past abnormalities or diseases of the following systems: gynecologic, cardiovascular, respiratory, gastrointestinal, hepatic, biliary, renal, endocrine/metabolic, musculoskeletal, hematologic/lymphatic, neurologic/psychiatric, dermatologic, immunologic, infectious disease, bleeding tendency, and allergy/drug sensitivity.

Demographics: Information about date of birth, gender, race/ethnicity, detailed smoking history and alcohol history will be recorded during screening.

Physical examination: Complete physical examinations will include examination of general appearance, skin, head, ears, eyes, nose, throat, heart, lungs, abdomen, lymph nodes, extremities, reproductive (if indicated based on symptomology and medical history) and nervous system and measurement of body weight. Abbreviated (targeted) physical examinations should include a cardiorespiratory assessment and abdominal exam but will focus on new symptoms and will include examination of relevant systems as identified by the investigator. Height and weight will be evaluated at screening and weight only thereafter.

ECOG performance status and vital signs: ECOG performance status will be recorded per the investigator's assessment of patient performance status. ECOG PS: 0=Fully active, able to carry on all pre-disease performance without restriction; 1=Restricted in physically strenuous activity but ambulatory and able to carry out work of a light or sedentary nature, e.g., light house work, office work; 2=Ambulatory and capable of all selfcare but unable to carry out any work activities; up and about more than 50% of waking hours; 3=Capable of only limited selfcare; confined to bed or chair more than 50% of waking hours; 4=Completely disabled; cannot carry on any selfcare; totally confined to bed or chair; 5=Dead. Vital signs will include body temperature, respiratory rate, heart rate, and systolic and diastolic blood pressures. Blood pressure and heart rate will be measured after 5 minutes in a sitting or semi-recumbent position by means of oscillometry, using a blood pressure measuring device. If systolic blood pressure is below 100 mmHg or above 150 mmHg and/or diastolic pressure is below 50 mmHg or above 90 mmHg, measurement will be repeated. The heart rate measurement will be repeated when below 50 beats per minutes (bpm) or above 100 bpm. If the measurement is still outside normal ranges, it is up to the investigator to judge if the measurement should be repeated. Vital sign measurements outside normal ranges will be assessed as ‘abnormal, not clinically significant’, or ‘abnormal, clinically significant’ by the investigator. In the latter case, the abnormal vital sign measurement will be reported as an AE and further investigated as clinically indicated.

Electrocardiograms: A single standard 12-lead ECG will be conducted at screening, and at one timepoint after VV1 administration. If the ECG is abnormal, it must be repeated. ECGs should thereafter be conducted as clinically indicated and per standard practice. The ECG parameters to be documented are as follows: rhythm, PR interval, heart rate, QRS interval, QT interval, QTcF interval, and QRS axis. ECGs will be recorded while the patient is resting in a supine or in a semi-recumbent position. ECGs will be read at site and categorized as ‘normal’, ‘abnormal, not clinically significant’, or ‘abnormal, clinically significant’.

Pharmacokinetic assessments: Viral PK will be assessed via RT-PCR of VSV-IFNβ-NIS. The first whole blood sample collection (Baseline) will be done at any time in the first 24 hours before VV1 administration. Cemiplimab PK in serum will be assessed via ELISA. Blood samples will be drawn pre-dose (generally within 2 hours prior to study treatment) and end of infusion for the first cycles and less frequently thereafter.

Tumor biopsies: All patients will have a baseline FFPE tissue identified for correlative research at the time of screening. Additionally, a mandatory fresh pre-treatment core biopsy (fine-needle aspirate is not adequate) is required for at least 3 out of 10 patients in Stage 1 of each of the IV melanoma cohorts (doublet and triplet therapy), all patients in Stage 2 of these IV melanoma cohorts, and all patients in both Stage 1 and Stage 2 of the V/IT melanoma cohort. The pre-treatment biopsy must be obtained within 28 days before enrollment and will be sent to a central lab for correlative research, performed retrospectively. All patients required to provide a fresh pre-treatment biopsy sample are also required to undergo on-treatment biopsies of the same lesion on Cycle 1 Day 3-8 and on Cycle 2 Day 1. An additional optional biopsy of any other lesion can be performed if patient provides consent. In case the originally biopsied lesion has become too small for repeat biopsy, another lesion may be biopsied instead. For all cohorts in both Stage 1 and Stage 2, upon disease progression at EOT, an additional optional biopsy of a progressing lesion can be performed if patient provides consent. IHC on current FFPE biopsies will be used to assess markers including PD-L1, CD3+ T-cells, and CD8+ T-cells. Other markers, including TIM3, LAG3, and IDO1 expression as well as cytotoxic cells, NK cells, Th-1 cells, or other immune cell markers in the injected tumor microenvironments may be evaluated. The IHC results will be compared to baseline.

Efficacy Assessments: RECIST 1.1 will be used throughout this study for assessment of tumor response. While either CT or MRI may be utilized, as per RECIST 1.1, CT is the preferred imaging technique in this study. The same imaging modality will have to be used for each patient throughout the duration of the study. Digital photographs will be used for assessment, per RECIST 1.1, for cutaneous melanoma lesions for Melanoma cohorts. Imaging assessment will be performed until PD. A radiological assessment of a CR or PR requires confirmatory imaging at least 4 weeks after the initial assessment of response was observed. The assessments for DCR, DOR and PFS will be based on RECIST measurements. The time of first study treatment administration to the first documented disease progression or death will determine PFS. The time of first observed response to the first documented disease progression or death will determine DOR. The OS will be estimated from the time of first study treatment administration. Patients will be followed every 3 months after end of study treatment.

Table 4 outlines the requirements for tumor assessment at baseline.

TABLE 4
Baseline eligibility tumor assessment

| | |
|---|---|
| Measurable | Tumor lesions: Must be accurately measured in at least one dimension (longest |
| Disease: | diameter in the plane of measurement is to be recorded) with a minimum size of: |
| | 10 mm by CT by computerized tomography (CT scan slice thickness no greater |
| | than 5 mm). |
| | 10 mm caliper measurement by clinical exam (lesions that cannot be accurately |
| | measured with calipers should be recorded as non-measurable). |
| | 20 mm by chest x-ray. |
| | Malignant lymph nodes: To be considered pathologically enlarged and |
| | measurable, a lymph node must be > 15 mm in short axis when assessed by CT |
| | scan. At baseline and in follow-up, only the short axis will be measured and |
| | followed. |
| | Skin lesions: Documentation by digital color photography, including a ruler to |
| | estimate the size of the lesion, is recommended. |
| Non- | All other lesions, including small lesions (longest diameter <10 mm or pathological |
| Measurable | lymph nodes with >10 to <15 mm short axis) as well as truly non-measurable |
| Disease: | lesions. Lesions considered truly non-measurable include: leptomeningeal disease, |
| | ascites, pleural or pericardial effusion, inflammatory breast disease, and |
| | lymphangitic involvement of skin or lung, abdominal masses, abdominal |
| | organomegaly identified by physical exam that is not measurable by reproducible |
| | imaging techniques. |
| Target | The most reproducible measurable lesions, up to a maximum of 2 lesions per organ |
| Lesions: | and 5 lesions in total, representative of all involved organs should be identified as |
| | target lesions and recorded and measured at baseline. |
| | Target lesions should be selected on the basis of their size (lesions with the longest |
| | diameter), should be representative of all involved organs, and, in addition, should |
| | be those that lend themselves to reproducible repeated measurements. |
| | Pathological nodes which are defined as measurable and that may be identified as |
| | target lesions must meet the criterion of a short axis of ≥ 15 mm. All target lesions |
| | will be calculated and reported as the baseline sum diameters. If lymph nodes are |
| | to be included in the sum, then as noted above, only the short axis is added into the |
| | sum. The baseline sum diameters will be used as reference to further characterize |
| | any objective tumor response. |
| Non-Target | All other lesions (or sites of disease) are identified as non-target lesions (chosen |
| Lesions: | based on their representativeness of involved organs and the ability to be |
| | reproduced in repeated measurements) and should be recorded at baseline. |
| | Measurements of these lesions are not required, but the presence or absence of |
| | each should be noted throughout follow-up. Lymph nodes with short axis > 10 mm |
| | but < 15 mm should be considered non-target lesions. Nodes that have a short axis < |
| | 10 mm are considered non-pathological and are not recorded or followed. |

On-study tumor assessments, evaluation of target lesions will be as follows:

Complete Response (CR): Disappearance of all target lesions. Any pathological lymph nodes (whether target or non-target) must have reduction in short axis to <10 mm. Tumor marker results must have normalized.

Partial Response (PR): At least a 30% decrease in the sum of diameters of target lesions, taking as reference the baseline sum diameters.

Stable Disease (SD): Neither sufficient shrinkage to qualify for PR nor sufficient increase to qualify for PD, taking as reference the smallest (nadir) sum of diameters since the treatment started.

Progressive Disease (PD): At least a 20% increase in the sum of the diameters of target lesions, taking as reference the smallest (nadir) sum since the treatment started, or the appearance of one or more new lesions. Requires not only 20% increase, but absolute increase of a minimum of 5 mm over sum.

On-study tumor assessments, evaluation of non-target lesions will be as follows:

Complete Response (CR): Disappearance of all non-target lesions and normalization of tumor markers. All lymph nodes must be non-pathological in size (<10 mm short axis).

Stable Disease (SD): Persistence of one or more non-target lesions and/or persistence of tumor marker level above the normal limits.

Progressive Disease (PD): Appearance of one or more new lesions and/or unequivocal progression of existing non-target lesions. When the patient also has measurable disease there must be an overall level of substantial worsening in non-target disease such that, even in presence of SD or PR in the target disease, the overall tumor burden has increased sufficiently to merit discontinuation of therapy.

Table 5 outlines the method for assessing best overall response. When nodal disease is included in the sum of target lesions, and the nodes decrease to “normal” size (<10 mm), they may still have a measurement reported on scans. This measurement should be recorded even though the nodes are normal in order not to overstate progression, should it be based on increase in size of the nodes. As noted earlier, this means that patients with CR may not have a total sum of ‘zero’. If there is suspicion of disease progression based on clinical or laboratory findings before the scheduled assessment, an unscheduled assessment should be performed. Patients with a global deterioration of health status requiring discontinuation of treatment without objective evidence of disease progression at that time should be reported as “symptomatic deterioration”. Every effort should be made to document objective progression even after discontinuation of treatment.

TABLE 5
Evaluation of best overall response

| Target | Non-Target | New | Overall | |
|---|---|---|---|---|
| Lesions | Lesions | Lesions | response | |
| CR | CR | No | CR | CR: disappearance of all target and |
| CR | Non-CR/non- | No | PR | non-target lesions, normalization of |
| | PD | | | |
| CR | NE | No | PR | tumor marker level, and reduction |
| PR | Non-PD or NE | No | PR | in pathological lymph nodes short |
| SD | Non-PD or NE | No | SD | axis to <10 mm. |
| Not all | Non-PD | No | NE | PR: 30% decrease in the sum of |
| evaluated | | | | |
| PD | Any | Yes or No | PD | diameters of the target lesions |
| Any | PD | Yes or No | PD | |
| Any | Any | Yes | PD | PD: 20% increase in the sum of |
| | | | | diameters of the target lesions |
| | | | | compared to nadir. |

Adverse Events: An AE is any symptom, physical sign, syndrome, or disease that either emerges during the study or, if present at screening, worsens during the study, regardless of the suspected cause of the event. All medical and psychiatric conditions (except those related to the indication under study) present at screening will be documented. Changes in these conditions and new symptoms, physical signs, syndromes, or diseases should be noted during the rest of the study. Laboratory, vital signs and ECG abnormalities should also be recorded as AEs when considered clinically significant. AEs may be volunteered spontaneously by the patient, discovered as a result of general questioning by the study staff, or determined by physical examination. During each visit to the study clinic, the patient will be asked, “Have you experienced any problems since your last visit?” All AEs will be recorded.

Primary outcome measures/analyses, proportion of tumor response: The primary efficacy endpoint is the proportion of tumor responses (ORR) by RECIST 1.1 (investigator-assessed). Tumor response will be considered synonymous with success, unless specified otherwise. A confirmed tumor response is defined to be a CR, or PR noted as the objective status on two consecutive evaluations at least 4 weeks apart. Tumor response will be evaluated using all cycles of treatment. ORR will be calculated overall and by cohort/subgroup using RECIST 1.1. The proportion of successes will be estimated by the number of successes divided by the total number of evaluable patients in each cohort. Confidence intervals for the true success proportion will be calculated according to the approach of Duffy and Santner in each cohort (Duffy, 1987). If more than the target number of patients is accrued in an expansion cohort, the additional patients in that cohort will not be used in any decision making; however, they will be included in final endpoint estimates and confidence intervals.

Rationale: In this study, combination therapy with oncolytic VV1, the anti-PD-1 cemiplimab, and the anti-CTLA4 monoclonal antibody ipilimumab is expected to enhance IT CD8+ T-cell infiltration and activation to improve therapeutic response in patients whose cancers are resistant to an anti-PD-1/PD-L1 alone. In the present study, conducted in a melanoma patient population that has failed single agent CPI, it is expected that cell infection will expand the available endogenous anti-tumor T cell repertoire, increase the tumor cell antigenicity for T cell recognition, enhance antigen presentation, and ultimately convert a non-inflamed or immune-excluded phenotype to a highly inflamed phenotype.

### Example 2: Phase 2 Trial of Voyager V1 (Vesicular Stomatitis Virus Expressing NIS and Human Interferon Beta, VV1), in Combination with Cemiplimab in Patients with Select Solid Tumors

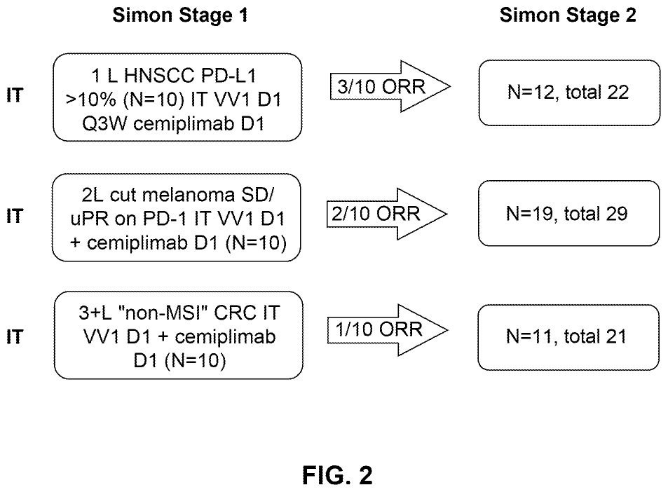

This example relates to an open label, non-randomized Phase 2 study designed to determine the anti-tumor activity and confirm the safety of VV1 in combination with cemiplimab (350 mg IV). This study is a variation of the study described in Example 1, wherein the hepatocellular, endometrial and NSCLC cohorts are closed, the melanoma cohort has been modified, and head and neck squamous cell carcinoma (HNSCC) and colorectal cancer (CRC) cohorts have been added. Accordingly, the study described in Example 2 includes melanoma, HNSCC, and CRC cohorts; and the study design is illustrated in FIG. 2.

The VSV-IFNβ-NIS as manufactured (i.e., undiluted) is in a buffer consisting of 5% sucrose, 50 mM Tris (pH 7.4), 2 mM MgCl₂. After manufacture, the virus is stored frozen at ≤−65° C. until it is prepared for use immediately prior to administration. The virus will be thawed and diluted in 100 mL of normal saline with 1% HSA and administered under close observation.

The VV1 dose will be administered as an intratumoral (IT) injection at 1.0×10⁹ TCID₅₀/mL (up to 5 mL total; in up to 5 lesions) on Day 1 (D1) in combination with IV cemiplimab (also given on D1). Data from an ongoing trial (VYR-VSV2-101) and investigator-initiated studies have demonstrated that doses of up to 1.7×10¹¹ TCID₅₀ (IV monotherapy and combination) and 3.0×10⁹ TCID₅₀ (IT in combination) are safe and potentially more efficacious than lower doses of VV1. Amplification of cemiplimab or VV1 toxicities is not expected based on experience to date with VV1 and other oncolytic viruses in combination with CPIs.

The IT route of VV1 administration offers the advantage of significantly reduced systemic toxicity, compared to IV VV1, with a more favorable safety profile without compromising antitumor activity. In the clinical development program, there have been no observations of sustained high circulating concentrations of virus-encoded IFNβ following IT VV1 administration. IT administration of VV1 is better tolerated than IV administration, with fewer systemic symptoms and fewer requirements for pre- and post-medication.

It is expected that combination therapy with oncolytic VSV-IFNβ-NIS and cemiplimab will enhance intratumoral CD8+ T-cell infiltration and activation to enhance therapeutic response in cancer patients who are previously resistant to CPI monotherapy. The trial will include correlative studies to monitor virus PK and pharmacodynamics (PD), including local and systemic immune responses, particularly monitoring of CD8+ T-cell responses. These studies will help establish the safety of combination therapy with VSV and checkpoint blockade; provide preliminary evidence of clinical efficacy in patients with advanced cancer; and elucidate the clinical mechanism of action and biomarkers of response to combination therapy.

The present study, conducted in a melanoma patient population that has failed single agent CPI, is designed to provide insight into putative mechanisms of re-sensitization to CPI through infusion of a single dose of oncolytic virus, with the hypothesis that cell infection will expand the available endogenous anti-tumor T cell repertoire, increase the tumor cell antigenicity for T cell recognition, enhance antigen presentation, and ultimately convert a non-inflamed or immune-excluded phenotype to a highly inflamed phenotype. Preliminary studies of immune responses in patients receiving VSV-IFNβ-NIS therapy suggest some patients develop T cell responses to viral antigens and known tumor antigens.

Study Objectives: A primary objective of the study is to determine the preliminary anti-tumor activity in each study cohort as measured by ORR both in the injected lesion(s) and according to standard criteria (RECIST 1.1) [Eisenhauer 2009]. Secondary objectives of the study include: determine the safety and tolerability of IT VV1 in combination with cemiplimab; evaluate the systemic PK of IT VV1 and cemiplimab; investigate the PD of IT VV1 in the blood by measuring serum IFNβ expression; and determine preliminary clinical activity of each cohort [e.g., PFS, DOR, DCR and OS].

Overall Study Design: Approximately 30-87 patients will be enrolled in the study. For the purposes of cohort expansion, a response is considered to be a 30% shrinkage in the injected lesion STL in the absence of RECIST 1.1 defined disease progression in the non-injected lesions. Patients will be enrolled into three parallel doublet cohorts, each one with an optimal Simon's two-stage design; in all cohorts (melanoma, HNSCC and CRC) patients will receive the same IT VV1/IV cemiplimab doublet. After cycle 1, cemiplimab will be dosed on D1 of each 3-week cycle. IT VV1 will be injected a second time on D1 of cycle 2 and, if feasible, IT VV1 may also be given repeatedly every 3 weeks (Q3W), until lack of clinical benefit or limiting toxicity. Efficacy evaluations will be conducted every 6 weeks (Q6W) (at the end of every other cycle).

Data from an ongoing trial (VYR-VSV2-101) and investigator-initiated studies have demonstrated that doses of up to 1.7×10¹¹ TCID₅₀ (IV monotherapy and combination) and 3.0×10⁹ TCID₅₀ (IT in combination) are safe and potentially more efficacious than lower doses of VV1 (see Section 1.1.4). The IT route of VV1 administration offers the advantage of significantly reduced systemic toxicity, compared to IV VV1, with a more favorable safety profile without compromising antitumor activity. In the clinical development program, there have been no observations of sustained high circulating concentrations of virus-encoded IFNβ following IT VV1 administration.

All enrolled patients on this study will only receive IT VV1 and thus will no longer be required to be hospitalized overnight on Day 1 and may be discharged for outpatient follow-up after 6 hours' observation, if clinically stable. Patients are then required to undergo outpatient safety evaluations on Days 2, 3, 4 and 5. Lab assessments performed on Days 2, 3, 4 and 5 must be evaluated and assessed for possible IFNβ toxicity on those days.

For all cohorts in the study an optimal Simon's two-stage design [Simon 1989] will be used. In the first stage, 10 evaluable patients will be accrued in each tumor cohort; the cohort will progress to the second stage based on a combination of safety and efficacy. If, in the first 10 patients, there are less than 3 objective responses in the HNSCC cohort, less than 2 responses in the melanoma cohort, or less than 1 response in the CRC cohort, that cohort will be stopped; otherwise, in the second stage 11-19 additional patients will be accrued for a total of 21-29 evaluable patients in each cohort.

Patients must have evaluable disease per RECIST 1.1. Patients must agree to provide a newly obtained biopsy prior to starting study treatment, and to repeat biopsies during study treatment, and to providing the acquired tissue, as well as other blood samples and buccal swabs, for biomarker analysis.

The NCI CTCAE, v5.0 will be used for grading overall toxicities and the ASTCT consensus grading will be used for grading CRS and neurologic toxicity [Lee 2019]. Safety assessments will include AEs, SAEs, PEs, vital sign measurements, ECOG status, clinical safety laboratory evaluations (hematology, serum chemistry and hepatic panels, coagulation and urinalysis) and ECGs.

For a given patient, the end of their participation usually corresponds to the safety visit 90 days after the last dose of cemiplimab. However, if the patient stopped treatment for reasons other than PD and efficacy assessments continued after the 90-day safety visit, the end of participation is the date of last study assessment.

Inclusion criteria: Patients meeting all of the following inclusion criteria at screening/Day 1 of first dosing will be eligible for participation in the study: (1) Age 18 years on day of signing informed consent. (2) Specific by tumor cohorts: (a) For the HNSCC cohort, histologically confirmed diagnosis of advanced and/or metastatic HNSCC suitable for first line immunotherapy: (i) HPV+ and HPV− patients are allowed; (ii) Primary tumor locations of oropharynx, oral cavity, hypopharynx, or larynx. Participants may not have a primary tumor site of nasopharynx (any histology); (iii) PD-L1 status of 10% per local CPS score. Samples should be provided to the central lab for post-hoc centralized testing; (iv) At least 12 months between last dose of prior adjuvant therapy and date of relapse diagnosis (if given); (v) No prior anti-PD-(L)1 treatment for HNSCC; (b) For the melanoma cohort, histologically confirmed diagnosis of advanced and/or metastatic cutaneous melanoma for which no existing options are considered to provide clinical benefit: (i) Best response of uPR, SD or PD to an anti-PD-(L)1-containing regimen; (ii) Prior anti-PD-(L)1 therapy must have lasted 12 weeks; (iii) Radiological progression was demonstrated during or after therapy with a PD-(L)1 immune CPI (only one prior line of PD-(L)1 therapy is permitted; (iv) If patient received anti-PD-1 as prior adjuvant therapy, patient should have relapsed during therapy or within the subsequent 6 months after last dose. Note: Progression on ipilimumab is not required; (v) Patients with BRAF V600-positive tumor(s) should have received prior treatment with a BRAF inhibitor (alone or in combination with a MEK inhibitor) in addition to treatment with an anti-PD-1 or to have declined targeted therapy. Note: Patients with BRAF V600-positive tumors with no clinically significant tumor-related symptoms nor evidence of rapidly progressive disease are not required to be treated with a BRAF inhibitor (alone or in combination with a MEK inhibitor) based on investigator's decision; (c) For the CRC cohort, a histologically confirmed diagnosis of advanced and/or metastatic CRC: (i) Received or are not eligible for standard of care fluoropyrimidine(s), oxaliplatin, irinotecan, anti-VEGF and EGFR-targeted therapies; (ii) Non-microsatellite instability high (non-MSI high); (iii) Progression on previous systemic therapy.

(3) At least one tumor lesion amenable to IT injection and biopsy that has not been previously irradiated. (a) If the lesion(s) to be injected has/have not previously been biopsied and confirmed as malignant, the clinical and/or radiological features of the lesion(s) must be unequivocally consistent with a malignant lesion. (4) Measurable disease based on RECIST 1.1, including; a 1 measurable lesion(s) to be injected. (5) Performance status of 0 or 1 on the ECOG Performance Scale. (6) Life expectancy of >3 months. (7) Willingness to provide biological samples required for the duration of the study, including a fresh tumor biopsy sample whilst on study. Notes: At baseline the sample can be fresh or archival with no intervening systemic therapy prior to enrollment. Tissue obtained for the biopsy must not be previously irradiated, but a new or progressing lesion in the radiation field is acceptable. (8) Adequate organ function defined as the laboratory values obtained ≤14 days prior to registration, as shown in Table 6.

TABLE 6

| System | Laboratory Value |
|---|---|
| Hematological | |
| ANC | ≥1500/mcL |
| Platelets | ≥100 000/mcL |
| Hemoglobin | ≥9 g/dL |
| Renal | |
| Serum creatinine OR Measured or | ≤2.0 × ULN OR ≥ 40 mL/min for |
| calculated creatinine clearance (CL) | patients with creatinine levels > |
| (per CKD-EPI) | 2.0 × ULN |
| Hepatic | |
| Albumin | ≥3.0 g/dL |
| Direct bilirubin | ≤1.5 × ULN |
| AST | ≤2.5 × ULN |
| ALT | ≤2.5 × ULN |

(9) Negative pregnancy test for female patients of childbearing potential. (10) Absence of active CNS involvement. Note: Pre-enrollment imaging of asymptomatic patients not mandatory. (11) Ability to provide written informed consent.

Exclusion criteria: Patients meeting any of the following exclusion criteria at screening/Day −1 of first dosing will not be enrolled in the study: (1) Availability of and patient acceptance of an alternative curative therapeutic option. (2) Recent or ongoing serious infection, including any active Grade 3 or higher per the NCI CTCAE, v5.0 viral, bacterial, or fungal infection within 2 weeks of registration. (3) Patients who have a diagnosis of ocular, mucosal or acral melanoma. (4) Known seropositivity for and with active infection with HIV; (a) Patients who are seropositive for HIV but are receiving antiviral therapy and show non-detectable viral load and a normal CD4 T cell count for at least 6 months are eligible.

(5) Seropositive for and with evidence of active viral infection with HBV. Patients who are HBsAg negative and HBV viral DNA negative are eligible; (a) Patients who had HBV but have received an antiviral treatment and show nondetectable viral DNA for 6 months are eligible. (b) Patients who are seropositive because of HBV vaccine are eligible. (6) Seropositive for and with active viral infection with HCV; (a) Patients who had HCV but have received an antiviral treatment and show no detectable HCV viral RNA for 6 months are eligible. (7) Known history of active or latent TB. (8) Any concomitant serious health condition, which, in the opinion of the investigator, would place the patient at undue risk from the study, including uncontrolled hypertension and/or diabetes, clinically significant pulmonary disease (e.g., chronic obstructive pulmonary disease requiring hospitalization within 3 months) or neurological disorder (e.g., seizure disorder active within 3 months).

(9) Prior therapy within the following timeframe before the planned start of study treatment as follows: (a) Small molecule inhibitors, and/or other investigational agent: ≤2 weeks or 5 halflives, whichever is shorter; (b) Chemotherapy, other monoclonal antibodies, antibody-drug conjugates, or other similar experimental therapies: ≤3 weeks or 5 half-lives, whichever is shorter; (c) Radioimmunoconjugates or other similar experimental therapies, 6 weeks or 5 half-lives, whichever is shorter. (10) NYHA classification Ill or IV, known symptomatic coronary artery disease, or symptoms of coronary artery disease on systems review, or known cardiac arrhythmias (atrial fibrillation or SVT). (11) Any known or suspected active organ-threatening autoimmune disease, such as inflammatory bowel disease, autoimmune hepatitis, lupus, or pneumonitis, with the exception of hypothyroidism and type 1 diabetes that are controlled with treatment. (12) Immunodeficiency or immunosuppression, including systemic corticosteroids at >10 mg/day prednisone or equivalent within 1 week prior to planned start of study treatment.

(13) History of Grade 3 or 4 immune-mediated adverse reaction to immune CPIs. (14) Toxicities from previous therapies that have not resolved to a Grade 1 or less. (15) History of non-infectious pneumonitis that required steroids, or current pneumonitis. (16) High volume disease, as assessed clinically by the medical monitor via parameters such as radiologic impression and tumor markers or LDH. (17) Portal vein thrombosis involving more than intrahepatic portal vein branches: thrombosis of the right or left portal vein branch or the bifurcation, partial or complete obstruction of the portal vein trunk. (18) Known concurrent malignancy. EXCEPTIONS: basal cell carcinoma of the skin, squamous cell carcinoma of the skin, or in-situ cervical cancer that has been treated with curative intent, prostate cancer confined to the prostate gland with Gleason score <6 or PSA <1, as well as any stage I cancer treated with curative intent or any prior cancer with a disease-free interval of ≥3 years. (19) Any other concurrent anti-cancer therapy (chemotherapy, immunotherapy, radiotherapy, or any ancillary therapy considered investigational [used for a non-FDA approved indication and in the context of a research investigation]).

(20) Has received a live vaccine within 30 days of planned start of study treatment. Seasonal influenza vaccines for injection are generally inactivated flu vaccines and are allowed. COVID-19 mRNA vaccines are also allowed, according to the following guidance: Patients in screening, who have not started study treatment, should schedule their vaccine to receive the 2nd dose at least 2 weeks prior to Day 1 (assuming vaccine is a 2-dose vaccine). If a single-dose vaccine is approved, the single dose should be at least 2 weeks before Day 1. Vaccination during Cycle 1 between the first and the second dose of cemiplimab is discouraged. Beginning with Cycle 2 a minimum of 7 days must elapse from last treatment to CoVID-19 vaccination and patients must have 7 days elapse from vaccination to next treatment on study. Efforts should be made to avoid treatment delays, but where necessary, a delay of up to 7 days in next treatment for the study drug will be permitted. Intranasal influenza vaccines (e.g., FLU-MIST) are live attenuated vaccines and are NOT allowed 4 weeks before first dose and 12 weeks after last dose of VV1 administration.

(21) Any of the following because this study involves an investigational agent whose genotoxic, mutagenic and teratogenic effect on the developing fetus and newborn are unknown: (a) WOCBP*, or sexually active men, who are unwilling to practice highly effective contraception prior to the initial dose/start of the first treatment prior to the start of the first treatment, during the study, and for at least 4 weeks after the last dose. Highly effective contraceptive measures include: stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device (IUD); intrauterine hormonereleasing system (IUS); bilateral tubal ligation; vasectomized partner; and or sexual abstinence †‡. (i) Stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; (ii) IUD; IUS; (iii) Bilateral tubal ligation; (iv) Vasectomized partner (provided that the male vasectomized partner is the sole sexual partner of the WOCBP study participant and that the vasectomized partner has obtained medical assessment of surgical success for the procedure); and/or sexual abstinencet†, ‡.

* WOCBP are defined as women who are fertile following menarche until becoming postmenopausal, unless permanently sterile. Permanent sterilization methods include hysterectomy, bilateral salpingectomy, and bilateral oophorectomy. A postmenopausal woman is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. Pregnancy testing and contraception are not required for women with documented hysterectomy or tubal ligation.

† Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.

‡ Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together.

(22) Pregnant or nursing women. (23) Men who are unwilling to use a condom (even if they have undergone a prior vasectomy) while having vaginal intercourse, while taking the drug and for 4 weeks after stopping treatment. (24) Prior Grade; a 3 toxicity on an anti-PD-1 therapy. (25) Intent to inject lesions with major vessel involvement (e.g., carotid artery, large hepatic vessels). (26) Has progressive disease within three (3) months of completion of curatively intended systemic treatment for locoregionally advanced head and neck cancer.

Dosage schedule and administration: Cycles are defined as 3 weeks (21 days). Both IT VV1 and cemiplimab are to be administered on Day 1 of Cycle 1 and on Day 1 of Cycle 2 onwards. VV1 IT injections should continue Q3W if feasible and cemiplimab infusions continue Q3W until limiting toxicity or lack of clinical benefit. End of treatment is defined as last dose of either study drug.

The treatment schedule for combination therapy is shown in Table 7. The VV1 dose is based on prior clinical experience and data (see Investigator Brochure). The cemiplimab dose is as recommended in package insert [Libtayo® 2020]. Cemiplimab should be given on Day 1 of each 21-day cycle for up to 2 years.

TABLE 7
Treatment schedule for VV1/cemiplimab combination

| Agent | Dose | Route | Day |
|---|---|---|---|
| Cemiplimab | 350 mg | IV | C1D1 then Q3W |
| | | | (C × D1) |

Observed for 60 minutes for IRR

| | | | |
|---|---|---|---|
| VV1 | 1 × 10⁹ TCID₅₀ (up to | IT | C1D1 then Q3W |
| | a total of 5 × 10⁹ | | (C × D1), if feasible |
| | across 1-5 lesions) | | |

Observed for 6 hours for virus-related systemic symptoms

VV1 IT injections should continue Q3W if feasible and cemiplimab infusions continue Q3W until limiting toxicity or lack of clinical benefit. End of treatment is defined as last dose of either study drug.

Cemiplimab administration: Cemiplimab should be prepared per the package insert [Libtayo® 2020]. Cemiplimab is administered prior to IT VV1 as an IV infusion over 30 minutes under close observation as per institutional standard of care. Patients should then be observed for 60 minutes for IRR, prior to receiving VV1. Cemiplimab should be given on Day 1 of each subsequent 21-day cycle for up to 2 years.

IT VVI dose administration: VV1 will be administered as described above on Day 1 to one or more tumor lesions using TB syringes (or equivalent) with 20- to 23-gauge needles. The needle type can also be per the investigator's discretion, to best distribute the virus within the lesion(s).

VV1 can be administered IT into multiple accessible lesions using an appropriate syringe and needle type for the location of the lesion(s). For VV1 treatment, 1-5 lesions may be injected; each tumor will be injected IT with at least 1.0×10⁹ TCID₅₀/mL, starting from largest to smallest, until a maximum volume of 5 mL (total 5.0×10⁹ TCID₅₀) is reached, or until all injectable lesions have been treated. Newly formed lesions should be treated first at the next cycle if the patient meets the retreatment criteria. Where there are only one or two large lesions, more than 1 mL can be given per tumor per physician's discretion and with agreement from the medical monitor.

Administration is permitted only directly into the tumor or accessible lymph node. If multiple lesions are designated for injection, the lesions should be at sites as disparate as possible in the body.

Once lesions are identified, the maximum volume to be injected is 5 mL distributed over different injectable tumors. Lesions must be greater than 1.0 cm in size at baseline to be eligible for IT VV1 administration. Distribution of volume of injection will be determined based on the size of lesion to be injected, as shown in Table 8.

TABLE 8
IT VV1 Administration General Guidelines

| Lesion size (longest dimension) | Maximum Injection Volume** |
|---|---|
| ≥5.0 cm | 5.0 mL |
| >2.5 cm to 5.0 cm | 2.0 mL |
| >1.0 cm to ≤2.5 cm | 1.0 mL |

For injection of >1 lesion contact the medical monitor
for discussion of dose distribution.

*Lesions≤1.0 cm can only be injected if clinically responding on prior VV1 injection. The treating physician has the discretion to identify and treat lesions, but they must be documented on the injection worksheet. VV1 will be prepared at a dose of 1.0×10⁹ TCID₅₀/mL. A maximum of 5.0×10⁹ TCID₅₀ in total can be given. Each tumor will be injected IT with at least 1.0×10⁹ TCID₅₀/mL, injecting between 3-5 or multiple lesions where applicable, starting from largest to smallest, until maximum volume of 5 mL (total 5.0×10⁹ TCID₅₀) is reached, or until all injectable lesions have been treated. Newly formed lesions should be treated first at the next cycle. Where there is only one or two large lesions, more than 1 mL can be given per tumor per physician's discretion. All concomitant measures for mitigation of virus-related systemic symptoms should be started prior to IT VV1 administration.

Patients may continue to receive IT VV1 treatment if the requirements shown in Table 9 are met.

TABLE 9
Requirements for continuation of study drugs

| Parameter | Week 6* | Week 12* |
|---|---|---|
| Injected lesion(s) response | Any | ≥30% shrinkage vs |
| | | baseline |
| RECIST response | SD, PR or CR | SD, PR or CR |
| excluding injected lesions | | |

| | |
|---|---|
| Safety of VV1 | Related G3 clinically significant AEs: |
| | interrupt until recovered to G0-1; recurrent |
| | G3 or G4 discontinue VV1 |
| Safety of cemiplimab | Dose adjustments according to CTCAE v5.0 |
| | Grade for cemiplimab-related AEs |

*Week 3-9 = Week 6; Week 12 = Week 10+

Descriptions of assessments: Medical history will be recorded during screening to ensure eligibility of the patients and will focus on relevant current or past abnormalities or diseases of the following systems: gynecologic, cardiovascular, respiratory, gastrointestinal, hepatic, biliary, renal, endocrine/metabolic, musculoskeletal, hematologic/lymphatic, neurologic/psychiatric, dermatologic, immunologic, infectious disease, bleeding tendency, and allergy/drug sensitivity.

Demographics: Information about date of birth, gender, race/ethnicity, detailed smoking history and alcohol history will be recorded during screening.

Physical examination: Complete physical examinations will include examination of general appearance, skin, head, ears, eyes, nose, throat, heart, lungs, abdomen, lymph nodes, extremities, reproductive (if indicated based on symptomology and medical history) and nervous system and measurement of body weight. Abbreviated (targeted) physical examinations should include a cardiorespiratory assessment and abdominal exam but will focus on new symptoms and will include examination of relevant systems as identified by the investigator. An AE report should be completed for all changes identified as clinically significant. Height and weight will be evaluated at screening and weight only thereafter.

ECOG performance status and vital signs: ECOG performance status will be recorded per the investigator's assessment of patient performance status. ECOG PS: 0=Fully active, able to carry on all pre-disease performance without restriction; 1=Restricted in physically strenuous activity but ambulatory and able to carry out work of a light or sedentary nature, e.g., light house work, office work; 2=Ambulatory and capable of all selfcare but unable to carry out any work activities; up and about more than 50% of waking hours; 3=Capable of only limited selfcare; confined to bed or chair more than 50% of waking hours; 4=Completely disabled; cannot carry on any selfcare; totally confined to bed or chair; 5=Dead. Vital signs will include body temperature, respiratory rate, heart rate, and systolic and diastolic blood pressures. Blood pressure and heart rate will be measured after 5 minutes in a sitting or semi-recumbent position by means of oscillometry, using a blood pressure measuring device. If systolic blood pressure is below 100 mmHg or above 150 mmHg and/or diastolic pressure is below 50 mmHg or above 90 mmHg, measurement will be repeated. The heart rate measurement will be repeated when below 50 beats per minutes (bpm) or above 100 bpm. If the measurement is still outside normal ranges, it is up to the investigator to judge if the measurement should be repeated. Vital sign measurements outside normal ranges will be assessed as ‘abnormal, not clinically significant’, or ‘abnormal, clinically significant’ by the investigator. In the latter case, the abnormal vital sign measurement will be reported as an AE and further investigated as clinically indicated.

Laboratory parameters: The following laboratory tests are to be performed: serology/virology, urinalysis, electrocardiograms (ECGs), drug administration, pharmacokinetic assessments, viral pharmacokinetics, cemiplimab pharmacokinetics, PD assessments, anti-VSV IgG assay, cemiplimab immunogenicity, serum IFN-beta and cytokine profile, peripheral blood mononuclear cells, tumor biopsies, efficacy assessments, and baseline assessments.

Serology and virology measurements are to be performed at screening, but recent previously reported results may be used for eligibility purposes at the Medical Monitor's discretion. HIV (1/2) testing is required if the patient has never been tested for HIV or testing was prior to primary cancer diagnosis or testing was performed; 12 months prior to study. Virology tests at screening are: HbsAg; HCV RNA; HIV-1/HIV-2 antibodies. Patients who had HIV, HBV or HCV infection and have received antiviral treatment, must demonstrate no detectable viral RNA/load for 6 months prior to enrolling in this study.

A standard dipstick urinalysis is sufficient for study purposes; however, sites may perform urinalysis according to their typical site practices. Any clinically significant abnormality should be investigated per standard medical practice and any AE diagnosed should be recorded.

A single standard 12-lead ECG will be conducted at screening, and at one timepoint after VV1 administration. If the ECG is abnormal, it must be repeated. ECGs should thereafter be conducted as clinically indicated and per standard practice. The ECG parameters to be documented are as follows: rhythm, PR interval, heart rate, QRS interval, QT interval, QTcF interval, and QRS axis. ECGs will be recorded while the patient is resting in a supine or in a semi-recumbent position. ECGs will be read at site and categorized as ‘normal’, ‘abnormal, not clinically significant’, or ‘abnormal, clinically significant’. If the ECG after study drug administration is determined to be abnormal, clinically significant, a corresponding AE report must be completed for the clinically significant finding.

Viral PK will be assessed via RT-PCR of VSV-IFNβ-NIS. The first whole blood sample collection (Baseline) will be done at any time in the first 24 hours before VV1 administration. Cemiplimab PK in serum will be assessed via ELISA. Blood samples will be drawn pre-dose (generally within 2 hours prior to study treatment) and end of infusion for the first cycles and less frequently thereafter.

Anti-VSV IgG assay is required to determine patient immune response to the oncolytic virus. IFNβ will be assessed in conjunction with the PK data. Other pro-inflammatory cytokines and those relevant to safety may be assayed on the same sample, as dictated by the safety profile and ongoing findings in the study. Peripheral blood mononuclear cells (PBMCs) will be collected at baseline and at specified time points post treatment for T-cell assays, including sequencing.

Tumor biopsies: All patients will have a baseline formalin-fixed, paraffin embedded (FFPE) tissue identified for correlative research at the time of screening. Additionally, a fresh pre-treatment core biopsy (fine-needle aspirate is not adequate) is requested if the patient has had systemic therapy since the prior biopsy. The pre-treatment biopsy tissue must not be previously irradiated, but a new or progressing lesion in the radiation field is acceptable. This pre-treatment biopsy is optional in stage 1 but is mandatory is stage 2 of the study. All patients are also required to undergo on-treatment biopsies of the same lesion per the schedule of assessments. An additional optional biopsy of any other lesion can be performed if patient provides consent. In case the originally biopsied lesion has become too small for repeat biopsy, another lesion may be biopsied instead. If no previously injected lesion is suitable for biopsy, record this information. For all cohorts in both Stage 1 and Stage 2, upon disease progression at EOT, an additional optional biopsy of a progressing lesion can be performed if patient provides consent. IHC on current FFPE biopsies will be used to assess markers including PD-L1, CD3+ T-cells, and CD8+ T-cells. Other markers, including TIM3, LAG3, and IDO1 expression as well as cytotoxic cells, NK cells, Th-1 cells, or other immune cell markers in the injected tumor microenvironments may be evaluated. The IHC results will be compared to baseline.

Efficacy assessments: Both isolated measurement of shrinkage of the injected lesion(s) and RECIST 1.1 will be used throughout this study for assessment of tumor response. While either CT or MRI may be utilized, as per RECIST 1.1, CT is the preferred imaging technique in this study. The same imaging modality will have to be used for each patient throughout the duration of the study. Digital photographs will be used for assessment, per RECIST 1.1, for cutaneous melanoma lesions. Imaging assessment will be performed until PD. A radiological assessment of a CR or PR requires confirmatory imaging at least 4 weeks after the initial assessment of response was observed. Injected lesion measurement will follow RECIST 1.1 but will only include the injected lesion(s) in the sum of target lesions. The assessments for OCR DOR and PFS will be based on RECIST measurements. The time of first study treatment administration to the first documented disease progression or death will determine PFS. The time of first observed response to the first documented disease progression or death will determine DOR. The OS will be estimated from the time of first study treatment administration. Patients will be followed every 3 months after end of study treatment.

Baseline eligibility tumor assessments will be conducted according to Table 10.

TABLE 10

| | |
|---|---|
| Measurable | Tumor lesions: Must be accurately measured in at least one |
| Disease: | dimension (longest diameter in the plane of measurement is to be |
| | recorded) with a minimum size of: |
| | 10 mm by CT by computerized tomography (CT scan slice |
| | thickness no greater than 5 mm). |
| | 10 mm caliper measurement by clinical exam (lesions that |
| | cannot be accurately measured with calipers should be |
| | recorded as non-measurable). |
| | 20 mm by chest x-ray. |
| | Skin lesions: Documentation by digital color photography, including |
| | a ruler to estimate the size of the lesion, is recommended. |
| | Malignant lymph nodes: To be considered pathologically enlarged |
| | and measurable, a lymph node must be > 15 mm in short axis when |
| | assessed by CT scan. At baseline and in follow-up, only the short |
| | axis will be measured and followed. |
| Non-Measurable | All other lesions, including small lesions (longest diameter <10 mm or |
| Disease: | pathological lymph nodes with >10 to <15 mm short axis) as well as |
| | truly non-measurable lesions. Lesions considered truly non- |
| | measurable include: leptomeningeal disease, ascites, pleural or |
| | pericardial effusion, inflammatory breast disease, and lymphangitic |
| | involvement of skin or lung, abdominal masses, abdominal |
| | organomegaly identified by physical exam that is not measurable by |
| | reproducible imaging techniques. |
| Target Lesions: | The most reproducible measurable lesions, up to a maximum of 2 |
| | lesions per organ and 5 lesions in total, representative of all involved |
| | organs should be identified as target lesions and recorded and |
| | measured at baseline. |
| | Target lesions should be selected on the basis of their size (lesions |
| | with the longest diameter), should be representative of all involved |
| | organs, and, in addition, should be those that lend themselves to |
| | reproducible repeated measurements. Pathological nodes which are |
| | defined as measurable and that may be identified as target lesions |
| | must meet the criterion of a short axis of ≥ 15mm. All target lesions |
| | will be calculated and reported as the baseline sum diameters. If |
| | lymph nodes are to be included in the sum, then as noted above, only |
| | the short axis is added into the sum. The baseline sum diameters will |
| | be used as reference to further characterize any objective tumor |
| | response. |
| Non-Target | All other lesions (or sites of disease) are identified as non-target |
| Lesions: | lesions (chosen based on their representativeness of involved organs |
| | and the ability to be reproduced in repeated measurements) and |
| | should be recorded at baseline. Measurements of these lesions are |
| | not required, but the presence or absence of each should be noted |
| | throughout follow-up. Lymph nodes with short axis > 10 mm but < |
| | 15 mm should be considered non-target lesions. Nodes that have a |
| | short axis < 10 mm are considered non-pathological and are not |
| | recorded or followed. |

On-study tumor assessments including evaluation of target and non-target lesions will be the same as set forth in Example 1 and Table 5.

Concomitant medication assessment: Any medication or blood product (including over-the-counter medications and supplements) used within 4 weeks prior to the first dose of investigational drug until 28 days (for all concomitant medications) or 90 days (for concomitant medications used to treat investigational drug-related AEs) following the last dose will be recorded, together with the main reason for its prescription. Any new anticancer treatments commenced within 90 days after the last dose of study drug should be recorded.

Primary outcome measures/analyses, proportion of tumor response: The primary efficacy endpoint is the proportion of tumor responses (ORR) in the injected lesion(s) and overall by RECIST 1.1 (investigator-assessed). Tumor response will be considered synonymous with success, unless specified otherwise. A confirmed tumor response is defined to be a CR, or PR noted as the objective status on two consecutive evaluations at least 4 weeks apart. Tumor response will be evaluated using all cycles of treatment. ORR will be calculated overall and by cohort/subgroup using RECIST 1.1. The proportion of successes will be estimated by the number of successes divided by the total number of evaluable patients in each cohort.

Efficacy parameters will include the following: DCR is defined as the percentage of patients with a CR, PR or SD for at least 2 consecutive tumor assessments (i.e., confirmed CR/PR or SD for; 12 weeks). DOR is defined for all evaluable patients who have achieved an objective response as the date at which the patient's earliest best objective status is first noted to be either a CR or PR to the earliest date progression is documented. DOR will be estimated using the method of Kaplan-Meier. The PFS is defined as the time from first dose of study drug to the earliest date documentation of disease progression or death due to any cause. The distribution of PFS will be estimated using the method of Kaplan-Meier [Kaplan and Meier 1958] overall and for appropriate subgroups and cohorts, using RECIST 1.1, as above. The percent progression-free for the 6, 12, 18, 24, etc. -month timepoints may also be reported. Survival time is defined as the time from first dose of study drug to death due to any cause. The distribution of survival time will be estimated using the method of Kaplan-Meier [Kaplan and Meier 1958] overall and for appropriate subgroups and cohorts, using RECIST 1.1, as above. The percent alive for the 6, 12, 18, 24, etc. -month timepoints may also be reported.

SEQUENCE LISTING: The sequence listing of the present application is submitted electronically as an ST.26 formatted xml file with a file name “11163_seqlist_ST26”, creation date of Nov. 23, 2022, and a size of 30.8. This sequence listing submitted is part of the specification and is hereby incorporated by reference in its entirety. The following sequences have a length that is below the minimum length permitted under ST.26 format: SEQ ID NO: 7 (Ala Ala Ser) and SEQ ID NO: 19 (Ala Ala Ser).

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### REFERENCES

ACS: American Cancer Society. Cancer Facts and FIGS. 2018 and 2019.
Antonia et al., N Engl J Med 2017; 377(20):1919-1929.
Arora et al., Adv Ther 2019; 36(10):2638-2678.
Barber, Viral Immunol 2004; 17(4):516-527.
Borcherding et al., J Mol Biol 2018; 430(14):2014-2029.
Bray et al., CA Cancer J Clin 2018; 68:394-424.
Buchbinder et al., Am J Clin Oncol. 201; 39(1):98-106.
Burtness et al., Ann Oncol 2018; 29:viii729.
Chaturvedi et al., J Clin Oncol 2011; 29(32):4294-301.
Chow et al, J Clin Oncol 2016; 34(32):3838-45.
Clinical Guidelines Network. Role of biological agents in first-line treatment of metastatic colorectal cancer, available at: https://wiki.cancer.org.au/australia/uidelines:Colorectal_cancer/Biological_aents_first-line_treatment_of_metastatic_CRC
Dranoff, Immunol Rev 2002; 188:147-54.
Duffy et al., Biometrics 1987; 43(1):81-93.
Durham et al., Mol Ther 2017; 25(8):1917-1932.
Eisenhauer et al., Eur J Cancer 2009; 45(2):228-47.
Garassino et al., Ann Oncol 2017; 28(suppl_2):ii28-ii51.
Garon et al., Lancet 2014; 384(9944):665-673.
Gentzler et al., Immunother 2016; 8(5):583-600. doi: 10.2217/imt-2015-0029.
Govindan et al., J Clin Oncol. 2017; 35(30):3449-3457. doi: 10.1200/JCO.2016.71.7629.
Hegde et al., Clin Cancer Res. 2016; 22(8):1865-1874.
Jakafi® US prescribing information available at https://www.jakafi.com/pdf/prescribing-information.pdf (1 Jul. 2021).
Jiang et al., EJNMMI Res 2017; 7(1):90.
Kaplan et al., J Am Statistical Assoc 1958; 53:457-481.
Kulangara et al., Arch Pathol Lab Med 2019; 143(3):330-7.
Kurisetty et al., Head Neck 2014; 36(11):1619-1627.
Lee et al., Int J Colorectal Dis. 2007; 22(6):699-704.
Lee et al., Biol Blood Marrow Transplant 2019; 25:625-638.
Levey et al., Ann Intern Med 2009; 150(9):604-612.
Libtayo® US prescribing information available at https://packageinserts.bms.com/pi/pi_yervoy.pdf (19 Oct. 2021).
Lichty et al., Trends Mol Med 2004; 10(5):210-216.
Long et al., J Clin Oncol 2016; 34(15):9568-9568.
Luchini et al., Ann Oncol 2019; 30(8):1232-1243
Luke et al., Nat Rev Clin Oncol 2017; 14(8):463-482.
Martini et al., World J Gastroenterol. 2017; 23(26):4675-4688.
Miller et al., Mol Ther Oncolytics 2014; 1:14005.
Miller et al., Expert Opin Biol Ther 2016; 16(1):15-32.
Miura et al., J Thoracic Oncol 2018; 13: S692 (P2.01-71).
Naik et al., Leukemia 2012; 26(8):1870-1878.
Obuchi et al., J Virol 2003; 77(16):8843-56.
O'Day et al., Ann Oncol. 2010; 21(8):1712-1717.
Patel et al., Oncotarget 2015; 6(32):33165-33177.
Puzanov et al., J Clin Oncol 2016; 34(22):2619-26.
Ramalingam et al., J Clin Oncol 2020; 38(15_suppl):9500-9500.
Reck et al., ESMO Open 2021; 6(5):100273.
Reck et al., N Engl J Med 2016; 375(19):1823-1833.
Ribas, Cancer Discov 2015; 5:915-919.
Ribas et al., Cell 2017; 170(6):1109-1119.
Rischin et al., J Clin Oncol (ASCO Meeting Abstracts) 2019; 37(15_suppl). Abstract 6000.
Rojas et al., Clin Cancer Res. 2015; 21(24):5543-51
Saloura et al., Hum Gene Ther 2010; 21(1):51-64.
Sampson et al., J Allergy Clin Immunol 2006; 117(2):391-397.
Sezer et al., Lancet 2021; 397(10274):592-604.
Shen W et al., Blood 2016; 127(11):1449-58.
Siegel et al., CA Cancer J Clin. 2019; 69(1):7-34.
Siegel et al., CA Cancer J Clin 2021; 71:7-33.
Simon, Control Clin Trials. 1989; 10(1):1-10.
Spranger et al., Sci Transl Med 2013; 5(200):200ra116.
Spigel et al. Impower110: Interim overall survival (OS) analysis of a Phase Ill study of atezolizumab (atezo) vs platinum-based chemotherapy (chemo) as first-line (1L) treatment (tx) in PD-L1-selected NSCLC. Presented at ESMO 2019; Sep. 27-Oct. 1, 2019; Barcelona, Spain. Abstract LBA78.
Sung et al., CA Cancer J Clin. 2021 May; 71(3):209-249.
Van, Oncologist. 2006; 11(9):1010-1017.
Willmon et al., Cancer Res 2009; 69(19):7713-7720.
Wolchok et al., J Clin Oncol 2021; 39(15_suppl):9506-9506.
Yervoy® US prescribing information available at https://packgeinrserts.bms.com/pi/pi_yervoy.pdf (19 Oct. 2021).
Yoo et al., Clin Colorectal Cancer. 2006; 6(3):202-207.
Zhang et al., Mol Ther 2016; 24(12):2109-17.

We claim:

1. A method of treating or inhibiting the growth of a tumor, comprising:
(a) selecting a patient with a cancer; and
(b) administering to the patient in need thereof a combination therapy comprising:
(i) a therapeutically effective amount of an oncolytic virus; and
(ii) a therapeutically effective amount of an antibody or antigen-binding fragment thereof that binds specifically to programmed death 1 (PD-1), wherein the anti-PD-1 antibody or antigen-binding fragment thereof comprises three heavy chain complementarity determining regions (HCDR1, HCDR2 and HCDR3) of a heavy chain variable region (HCVR) comprising the amino acid sequence of SEQ ID NO: 1 and three light chain complementarity determining regions (LCDR1, LCDR2 and LCDR3) of a light chain variable region (LCVR) comprising the amino acid sequence of SEQ ID NO: 2.

2. The method of claim 1, wherein the oncolytic vesiculovirus comprises an oncolytic vesicular stomatitis virus (VSV).

3. The method of claim 2, wherein the VSV comprises a recombinant VSV.

4. The method of claim 3, wherein the recombinant VSV expresses a cytokine.

5. The method of claim 4, wherein the cytokine comprises an interferon-beta (IFNb).

6. The method of claim 5, wherein a nucleic acid sequence encoding the IFNb is positioned between M and G vial genes.

7. The method of any one of claims 3-6, wherein the recombinant VSV further expresses a sodium/iodide symporter (NIS).

8. The method of claim 7, wherein a nucleic acid sequence encoding the NIS is positioned between G and L viral genes.

9. The method of any one of claims 1-8, wherein the oncolytic virus is Voyager V1.

10. The method of any one of claims 1-9, wherein the anti-PD-1 antibody or antigen-binding fragment thereof comprises HCDR1, HCDR2, HCDR3, LCDR1, LCDR2, and LCDR3 comprising the amino acid sequences of SEQ ID NOs: 3, 4, 5, 6, 7, and 8, respectively.

11. The method of any one of claims 1-11, wherein the anti-PD-1 antibody or antigen-binding fragment thereof comprises HCVR comprising the amino acid sequence of SEQ ID NO: 1; and LCVR comprising the amino acid sequence of SEQ ID NO: 2.

12. The method of any one of claims 1-11, wherein the anti-PD-1 antibody or antigen-binding fragment thereof comprises a heavy chain comprising the amino acid sequence of SEQ ID NO: 9 and a light chain comprising the amino acid sequence of SEQ ID NO: 10.

13. The method of any one of claims 1-12, wherein the anti-PD-1 antibody or antigen-binding fragment thereof comprises cemiplimab or a bioequivalent thereof.

14. The method of any one of claims 1-13, wherein the oncolytic virus is administered to the patient in one or more doses of 10 to 10¹⁴ TCID₅₀.

15. The method of claim 14, wherein the oncolytic virus is administered to the patient in one or more doses of 10⁹ TCID₅₀ or 10¹¹ TCID₅₀.

16. The method of claim 13 or 14, wherein one dose of 10⁹ TCID₅₀ oncolytic virus is administered to the patient, and another dose of 10¹¹ TCID₅₀ oncolytic virus is administered to the patient.

17. The method of any one of claims 1-16, wherein the anti-PD-1 antibody or antigen-binding fragment thereof is administered to the patient in one or more doses of about 0.1 mg/kg to about 20 mg/kg of body weight of the patient.

18. The method of any one of claims 1-17, wherein the anti-PD-1 antibody or antigen-binding fragment thereof is administered to the patient in one or more doses of about 1 mg to about 1000 mg.

19. The method of claim 18, wherein the anti-PD-1 antibody or antigen-binding fragment thereof is administered to the patient in one or more doses of 350 mg.

20. The method of any one of claims 1-19, wherein the oncolytic virus is administered to the patient intratumorally or intravenously.

21. The method of any one of claims 1-20, wherein the anti-PD-1 antibody or antigen-binding fragment thereof administered to the patient intravenously, subcutaneously or intraperitoneally.

22. The method of any one of claims 1-21, wherein the method further comprises administering a therapeutically effective amount of a CTLA-4 inhibitor comprising ipilimumab or a bioequivalent thereof.

23. The method of claim 22, wherein the CTLA-4 inhibitor is ipilimumab.

24. The method of claim 22 or 23, comprising concurrently administering one or more doses of the oncolytic virus and one or more doses of the CTLA-4 inhibitor.

25. The method of any one of claims 22-24, comprising administering one or more doses of the oncolytic virus and one or more doses of the CTLA-4 inhibitor before administering one or more doses of the anti-PD-1 antibody or antigen-binding fragment thereof.

26. The method of any one of claims 22-25, comprising administering one or more doses of the oncolytic virus and one or more doses of the CTLA-4 inhibitor 7 days before administering one or more doses of the anti-PD-1 antibody or antigen-binding fragment thereof.

27.

28. The method of any one of claims 22-27, wherein the CTLA-4 inhibitor is administered to the patient in one or more doses of about 0.1 mg/kg to about 15 mg/kg of body weight of the patient.

29. The method of any one of claims 22-28, wherein the CTLA-4 inhibitor is administered to the patient in one or more doses of about 1 mg to about 600 mg.

30. The method of claim 29, wherein the CTLA-4 inhibitor is administered to the patient in one or more doses of 50 mg.

31. The method of any one of claims 22-30, wherein the CTLA-4 inhibitor is administered to the patient intravenously, subcutaneously or intraperitoneally.

32. The method of any one of claims 1-29, wherein the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

33. The method of any one of claims 1-30, wherein administration of the combination therapy leads to at least one therapeutic effect selected from: reduction in tumor cell number, tumor regression, partial response, and complete response.

34. The method of any one of claims 1-33, wherein administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with a monotherapy selected from: the oncolytic virus and the anti-PD-1 antibody or antigen-binding fragment thereof.

35. The method of claim 34, wherein administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with oncolytic virus monotherapy.

36. The method of any one of claims 1-35, wherein the tumor growth is inhibited by at least 50% as compared to an untreated patient.

37. The method of any one of claims 22-33, wherein administration of the combination therapy leads to at least one improvement selected from: abscopal effect, delay in tumor growth, increase in overall survival, increase in progression free survival, increase in overall response rate, increase in complete response, increase in partial response, and increase in stable disease, as compared to patients treated with a monotherapy selected from: the oncolytic virus, the anti-PD-1 antibody or antigen-binding fragment thereof, and the CTLA-4 inhibitor.

38. The method of claim 37, wherein the tumor growth is inhibited by at least 50% as compared to a patient administered the oncolytic virus, the anti-PD-1 antibody or antigen-binding fragment thereof, or the CTLA-4 inhibitor as monotherapy.

39. The method of claim 37 or 38, wherein the tumor growth is inhibited by at least 50% as compared to a patient administered any two of the oncolytic virus, the anti-PD-1 antibody or antigen-binding fragment thereof, and the CTLA-4 inhibitor.

40. The method of any one of claims 1-39, further comprising administering an additional therapeutic agent or therapy to the patient.

41. The method of claim 40, wherein the additional therapeutic agent or therapy is selected from: radiation, surgery, a chemotherapeutic agent, a cancer vaccine, a B7-H3 inhibitor, a B7-H4 inhibitor, a lymphocyte activation gene 3 (LAG3) inhibitor, a T cell immunoglobulin and mucin-domain containing-3 (TIM3) inhibitor, a galectin 9 (GAL9) inhibitor, a V-domain immunoglobulin (Ig)-containing suppressor of T-cell activation (VISTA) inhibitor, a Killer-Cell Immunoglobulin-Like Receptor (KIR) inhibitor, a B and T lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor (GM-CSF), a cytotoxin, an interleukin 6 receptor (IL-6R) inhibitor, an interleukin 4 receptor (IL-4R) inhibitor, an IL-10 inhibitor, IL-2, IL-7, IL-12, IL-21, IL-15, an antibody-drug conjugate, an anti-inflammatory drug, and combinations thereof.

42. A combination of an oncolytic virus and an antibody or antigen-binding fragment thereof that binds specifically to programmed death 1 (PD-1), wherein the combination is for use in a method of treating or inhibiting the growth of a tumor, the method comprising:
(a) selecting a patient with a cancer; and
(b) administering to the patient in need thereof a combination therapy comprising:
(i) a therapeutically effective amount of an oncolytic virus; and
(ii) a therapeutically effective amount of an antibody or antigen-binding fragment thereof that binds specifically to programmed death 1 (PD-1), wherein the anti-PD-1 antibody or antigen-binding fragment thereof comprises three heavy chain complementarity determining regions (HCDR1, HCDR2 and HCDR3) of a heavy chain variable region (HCVR) comprising the amino acid sequence of SEQ ID NO: 1 and three light chain complementarity determining regions (LCDR1, LCDR2 and LCDR3) of a light chain variable region (LCVR) comprising the amino acid sequence of SEQ ID NO: 2.

43. A combination of an oncolytic virus, an antibody or antigen-binding fragment thereof that binds specifically to programmed death 1 (PD-1), and a CTLA-4 inhibitor comprising ipilimumab or a bioequivalent thereof, wherein the combination is for use in a method of treating or inhibiting the growth of a tumor, the method comprising:
(a) selecting a patient with a cancer; and
(c) administering to the patient in need thereof a combination therapy comprising:
(i) a therapeutically effective amount of an oncolytic virus;
(ii) a therapeutically effective amount of an antibody or antigen-binding fragment thereof that binds specifically to programmed death 1 (PD-1), wherein the anti-PD-1 antibody or antigen-binding fragment thereof comprises three heavy chain complementarity determining regions (HCDR1, HCDR2 and HCDR3) of a heavy chain variable region (HCVR) comprising the amino acid sequence of SEQ ID NO: 1 and three light chain complementarity determining regions (LCDR1, LCDR2 and LCDR3) of a light chain variable region (LCVR) comprising the amino acid sequence of SEQ ID NO: 2; and
(iii) a therapeutically effective amount of a CTLA-4 inhibitor comprising ipilimumab or a bioequivalent thereof.

44. A kit comprising an oncolytic virus and an antibody or antigen-binding fragment thereof that binds specifically to programmed death 1 (PD-1), in combination with written instructions for use of a therapeutically effective amount of a combination of the oncolytic virus and the anti-PD-1 antibody or antigen-binding fragment thereof for treating or inhibiting the growth of a tumor of a patient.

45. A kit comprising an oncolytic virus, an antibody or antigen-binding fragment thereof that binds specifically to programmed death 1 (PD-1), and a CTLA-4 inhibitor comprising ipilimumab or a bioequivalent thereof, in combination with written instructions for use of a therapeutically effective amount of a combination of the oncolytic virus, the anti-PD-1 antibody or antigen-binding fragment thereof, and the CTLA-4 inhibitor for treating or inhibiting the growth of a tumor of a patient.


SEQUENCE LISTING (25 sequences)

SEQ ID NO: 1 - protein, 117 aa, synthetic construct [sequence omitted]

SEQ ID NO: 2 - protein, 107 aa, synthetic construct [sequence omitted]

SEQ ID NO: 3 - protein, 8 aa, synthetic construct
  REGION 1..8: note = Synthetic
GFTFSNFG

SEQ ID NO: 4 - protein, 8 aa, synthetic construct
  REGION 1..8: note = Synthetic
ISGGGRDT

SEQ ID NO: 5 - protein, 10 aa, synthetic construct
  REGION 1..10: note = Synthetic
VKWGNIYFDY

SEQ ID NO: 6 - protein, 6 aa, synthetic construct
  REGION 1..6: note = Synthetic
LSINTF

SEQ ID NO: 7 - skipped

SEQ ID NO: 8 - protein, 9 aa, synthetic construct
  REGION 1..9: note = Synthetic
QQSSNTPFT

SEQ ID NO: 9 - protein, 444 aa, synthetic construct
  REGION 1..444: note = Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, synthetic construct
  REGION 1..120: note = Synthetic
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 12 - protein, 108 aa, synthetic construct
  REGION 1..108: note = Synthetic
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

SEQ ID NO: 13 - protein, 116 aa, synthetic construct
  REGION 1..116: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSS

SEQ ID NO: 14 - protein, 108 aa, synthetic construct
  REGION 1..108: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIK

SEQ ID NO: 15 - protein, 8 aa, synthetic construct
  REGION 1..8: note = Synthetic
GFTFSNYE

SEQ ID NO: 16 - protein, 8 aa, synthetic construct
  REGION 1..8: note = Synthetic
IRTSGTTK

SEQ ID NO: 17 - protein, 9 aa, synthetic construct
  REGION 1..9: note = Synthetic
AGGGTFLHY

SEQ ID NO: 18 - protein, 6 aa, synthetic construct
  REGION 1..6: note = Synthetic
QGIASY

SEQ ID NO: 19 - skipped

SEQ ID NO: 20 - protein, 10 aa, synthetic construct
  REGION 1..10: note = Synthetic
QQAKSFPMYT

SEQ ID NO: 21 - protein, 446 aa, synthetic construct
  REGION 1..446: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 215 aa, synthetic construct
  REGION 1..215: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 24 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 25 - protein, 643 aa, Homo sapiens
MEAVETGERPTFGAWDYGVFALMLLVSTGIGLWVGLARGGQRSAEDFFTGGRRLAALPVGLSLSASFMSAVQVLGVPSEAYRYGLKFLWMCLGQLLNSVLTALLFMPVFYRLGLTSTYEYLEMRFSRAVRLCGTLQYIVATMLYTGIVIYAPALILNQVTGLDIWASLLSTGIICTFYTAVGGMKAVVWTDVFQVVVMLSGFWVVLARGVMLVGGPRQVLTLAQNHSRINLMDFNPDPRSRYTFWTFVVGGTLVWLSMYGVNQAQVQRYVACRTEKQAKLALLINQVGLFLIVSSAACCGIVMFVFYTDCDPLLLGRISAPDQYMPLLVLDIFEDLPGVPGLFLACAYSGTLSTASTSINAMAAVTVEDLIKPRLRSLAPRKLVIISKGLSLIYGSACLTVAALSSLLGGGVLQGSFTVMGVISGPLLGAFILGMFLPACNTPGVLAGLGAGLALSLWVALGATLYPPSEQTMRVLPSSAARCVALSVNASGLLDPALLPANDSSRAPSSGMDASRPALADSFYAISYLYYGAPGTLTTVLCGALISCLTGPTKRSTLAPGLLWWDLARQTASVAPKEEVAILDDNLVKGPEELPTGNKKPPGFLPTNEDRLFFLGQKELEGAGSWTPCVGHDGGRDQQETNL